(19) United States
(12) Patent Application Publication
(10) Pub. No.: US 2024/0325169 A1
(43) Pub. Date: Oct. 3, 2024

(54) Femoral Component Extractor

(71) Applicant: Tri-Sphere Holdings, LLC, Osprey, FL (US)

(72) Inventor: Dana Andrew Alden, Osprey, FL (US)

(73) Assignee: Tri-Sphere Hodings, LLC, Osprey, FL (US)

(21) Appl. No.: 18/130,400

(22) Filed: Apr. 3, 2023

(51) Int. Cl. A61F 2/46

(52) U.S. Cl. CPC A61F 2/4607; A61F 2002/30405; A61F 2002/4619; A61F 2002/4628; A61F 2310/00017

(57) ABSTRACT

The invention is defined by the claims set forth herein; however, briefly, the invention herein is an extractor for a human femoral component with a trunnion neck comprising, a plurality of extractor sections, including a first section with a first axis, a second section with a second axis, and a third section with a third axis; a body with a threaded hole defined therein that is provided with a clamping body section and a central body section, a pivoting member with first end, a second end, and a pivot hole defined thereinbetween that includes a clamping structure located at the second end that is shaped to clamp the trunnion neck of the femoral component; and a pivot that secures the pivoting member to the body by extending through the pivot hole defined in the pivoting member and the pivot hole defined in the fulcrum structure of the body.

### CROSS-REFERENCE TO RELATED APPLICATIONS

This patent application is a continuation of U.S. patent application Ser. No. 16/900,612, now U.S. Pat. No. 11,617,660, the disclosure of which is incorporated herein by reference.

### BACKGROUND OF THE INVENTION

It has been known in orthopedic surgical practices to implant artificial hips. Such prosthetic devices include a femoral component and an acetabular component, which together function as a ball and socket joint. This patent application relates to surgical instruments used to extract the femoral component of an artificial hip. The femoral component is often fabricated from metallic biomaterials with a surface finish that is highly polished. The smooth surfaces of the femoral component inhibit corrosion and bacterial growth. FIG. 8 of the drawings provided herein depicts such a femoral component, and, as shown, the femoral component includes a stem provided with an axis and a spherically-shaped head that extends from the axis of the stem at an irregular angle (i.e. an angle that is not 90 degrees).

The stem is shaped to be inserted axially into a patient's femur. Naturally, before the femoral component can be implanted, the patient's existing femoral head must be removed and the femur prepared to receive the prosthesis. The surgeon accomplishes this by broaching a cavity within the femoral canal that is shaped according to the stem. Often, surgeons undersize the cavity and impact the femoral component into the femur so that the prosthesis is firmly secured without any voids where bacteria and other infection causing agents can grow. Alternatively, surgeons fill the cavity with a type of cement and then fix the stem of the femoral component within the cement.

Unfortunately, artificial implants loosen, components corrode and break, bio-compatibility degrades, and infections develop. Thus, patients with artificial hips sometimes require hip revision surgery. In such a procedure, the prosthetic implants must be removed, including the femoral component. However, as noted above, the femoral component is often well-fixed within the patient's femur. As noted above, the irregular geometric configuration combined with the polished surfaces render vice-grip instruments largely ineffective as they slip on the femoral component's smooth surfaces.

If the femoral component cannot be extracted, the surgeon must remove the femoral component surgically via an extended trochanteric osteotomy, a procedure that often has complications and extends patient recovery. Thus, there is a need for an extractor that can clamp onto the polished surfaces of the femoral component without slipping. There is also a need for an extractor that can remove a femoral component despite the irregular geometry associated with such prosthetic devices. There is also a need for an extractor that enables a surgeon to remove a well-fixed femoral component from a patient's femur without resorting to additional surgical procedures that have complications of their own and that extend a patient's recovery time.

The foregoing does not purport to be an exhaustive explication of all the disadvantages associated with prior art extractors; however, the present invention is directed to overcoming these (and other) disadvantages inherent in prior art systems. The advantages of the present invention will become readily apparent to those of ordinary skill in the art after reading the disclosure provided herein.

### BRIEF DESCRIPTION OF THE DRAWINGS

FIG. 1 depicts a perspective view from one side of the extractor clamping onto a femoral component.

FIG. 2 depicts a perspective view from one side of the extractor clamping onto a femoral component.

FIG. 3 depicts a cross-sectional view of the extractor in FIG. 2 clamping onto a femoral component.

FIG. 4 depicts a perspective view from one side of the extractor clamping onto a femoral component.

FIG. 5 depicts a cross-sectional view of the extractor in FIG. 4 clamping onto a femoral component.

FIG. 6 depicts a cross-sectional view of the extractor in FIG. 7 clamping onto a femoral component.

FIG. 7 depicts a perspective view of the extractor clamping onto a femoral component.

FIG. 8 depicts a perspective view of the extractor.

FIG. 9 depicts a perspective view of extractor.

FIG. 10 depicts a cross-sectional view of the extractor in FIG. 9.

FIG. 11 depicts a detailed view of the cross-sectional view of the extractor in FIG. 10.

FIG. 12 depicts a perspective view of the body of the extractor.

FIG. 13 depicts a perspective view of the body of the extractor.

FIG. 14 depicts a perspective view of the body of the extractor.

FIG. 15 depicts a perspective view of the body of the extractor.

FIG. 16 depicts a perspective view of the body of the extractor.

FIG. 17 depicts a cross-sectional view of the body of the extractor in FIG. 16.

FIG. 18 depicts a perspective view of the body of the extractor.

FIG. 19 depicts a cross-sectional view of the body of the extractor in FIG. 18.

FIG. 20 depicts a perspective view of a fastener included in the extractor.

FIG. 21 depicts a perspective view of a fastener included in the extractor.

FIG. 22 depicts a perspective view of the body of the extractor.

FIG. 23 depicts a cross-sectional view of the body of the extractor in FIG. 22.

FIG. 24 depicts a perspective view of the body of the extractor.

FIG. 25 depicts a cross-sectional view of the body of the extractor in FIG. 24.

FIG. 26 depicts a perspective view of the lever included in the extractor.

FIG. 27 depicts a detailed view of the lever in FIG. 26.

FIG. 28 depicts a perspective view of the lever included in the extractor.

FIG. 29 depicts a perspective view of the lever included in the extractor.

FIG. 30 depicts a cross-sectional view of the lever in FIG. 29.

FIG. 31 depicts a perspective view of the locking component included in the extractor.

FIG. 32 depicts a perspective view of the locking component included in the extractor.

FIG. 33 depicts a cross-sectional view of the locking component in FIG. 32.

FIG. 34 depicts a perspective view of the locking component included in the extractor.

FIG. 35 depicts a cross-sectional view of the locking component in FIG. 34.

FIG. 36 depicts a perspective view of the strike plate that is offset from the axis of the body.

FIG. 37 depicts a perspective view of the bracket included in the extractor.

FIG. 38 depicts a perspective view of the bracket included in the extractor.

FIG. 39 depicts a perspective view of the bracket included in the extractor.

FIG. 40 depicts a perspective view of the bracket included in the extractor.

FIG. 41 depicts a perspective view of the bracket included in the extractor.

FIG. 42 depicts a perspective view of the shaft included in the extractor.

FIG. 43 depicts a perspective view of the shaft included in the extractor.

FIG. 44 depicts a perspective view of the shaft included in the extractor.

FIG. 45 depicts a perspective view of the shaft included in the extractor.

FIG. 46 depicts a cross-sectional view of the shaft depicted in FIG. 44.

FIG. 47 depicts a perspective view of the strike plate included in the extractor.

FIG. 48 depicts a perspective view of the strike plate included in the extractor.

FIG. 49 depicts a cross-sectional view of the strike plate depicted in FIG. 48.

FIG. 50 depicts a perspective view of the extractor with the bracket configured to rotate while the jaws clamp a femoral component.

FIG. 51 depicts a cross-sectional view of the extractor depicted in FIG. 50.

FIG. 52 depicts a perspective view of the extractor with the bracket configured to rotate while the jaws clamp a femoral component.

FIG. 53 depicts a perspective view of the extractor with the bracket configured to lock in place while the jaws clamp a femoral component.

FIG. 54 depicts a cross-sectional view of the extractor depicted in FIG. 53.

FIG. 55 depicts a perspective view of the extractor with the bracket configured to rotate while the jaws clamp a femoral component.

FIG. 56 depicts a perspective view of the gear included with the angle selector, which is itself included with the extractor.

FIG. 57 depicts a detailed view of the gear depicted in FIG. 56.

FIG. 58 depicts a perspective view of the gear included with the angle selector, which is itself included with the extractor.

FIG. 59 is a perspective view of the mounting plate included with the extractor.

FIG. 60 is a perspective view of the mounting plate included with the extractor.

FIG. 61 is a perspective view of the mounting plate included with the extractor.

FIG. 62 is a perspective view of the mounting plate included with the extractor.

FIG. 63 is a perspective view of the tightener included with the extractor.

FIG. 64 is a perspective view of the tightener included with the extractor.

FIG. 65 is a perspective view of the male threaded fastener included with the extractor.

FIG. 66 is a perspective view of the thumb screw included with the extractor.

FIG. 67 is a perspective view of the femoral component.

FIG. 68 is a perspective view of the femoral component.

FIG. 69 is a perspective view of the femoral component.

### SUMMARY OF THE INVENTION

The invention is defined by the claims set forth herein; however, briefly, the invention herein is an extractor for a femoral component with a trunnion neck comprising, a.

### DETAILED DESCRIPTION OF THE INVENTION

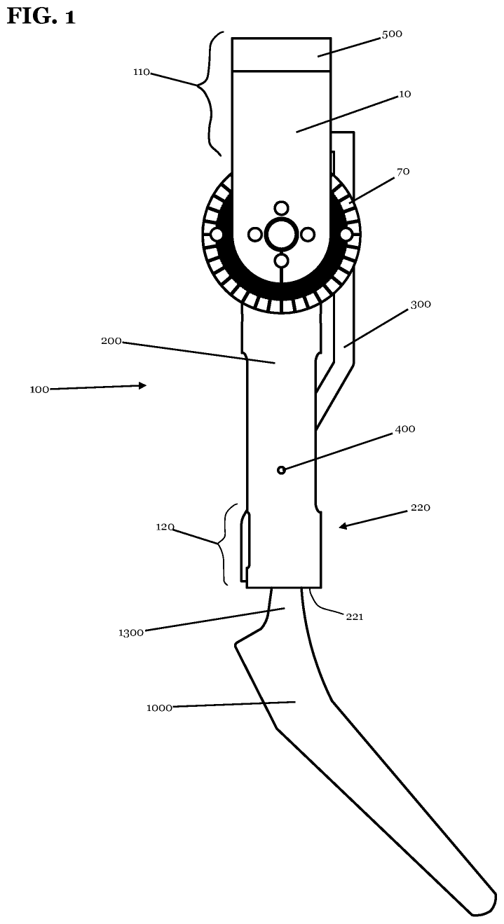

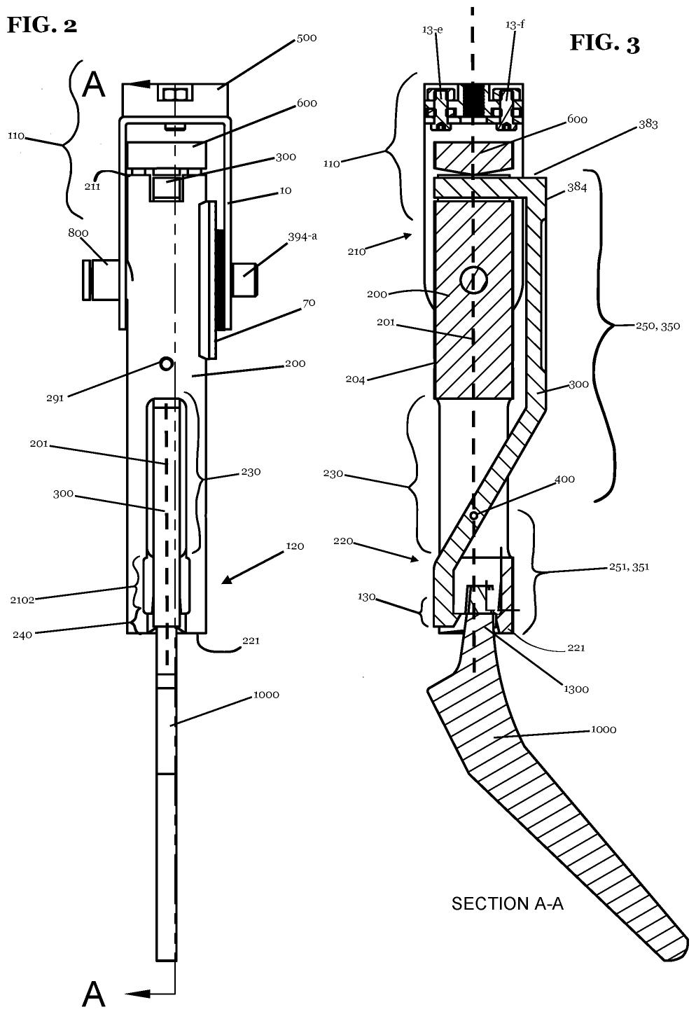

FIG. 1 depicts an extractor 100 constituting a presently preferred embodiment of the invention disclosed herein. The extractor 100 is provided with first end 110 and a second end 120, and, in FIG. 1, is depicted clamping a femoral component 1000. As used herein, the term “end” is defined to include the extreme end, as well as a portion extending from the extreme end. As FIG. 1 also depicts, the extractor 100 is provided with a bracket 10, a locking component 600 (shown in FIGS. 2 and 3), a fulcrum 400, a lever 300, a body 200, and an angle selector 70.

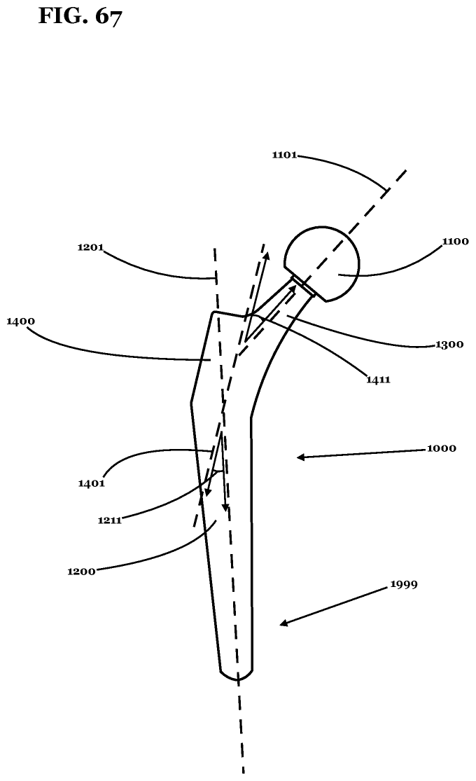

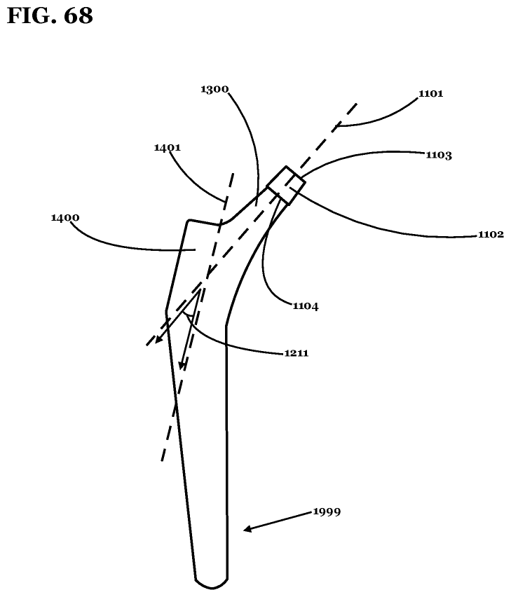

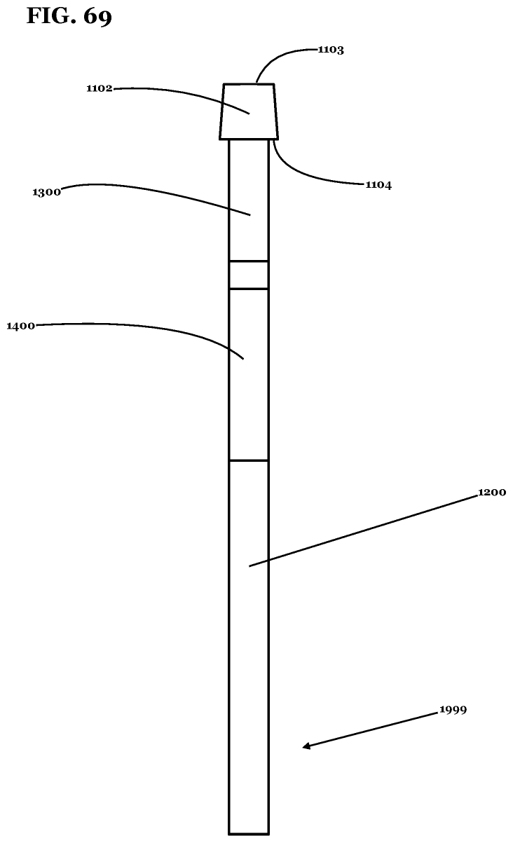

The femoral component referred to above is depicted in FIGS. 67, 68, and 69. As FIG. 67 illustrates, the femoral component 1000 is provided with a head 1100, a trunnion axis 1101, a stem 1200 (which includes a stem axis 1201), and a component end 1999. As FIGS. 67, 68, and 69 further show, the head 1100 (shown in FIG. 67) is disposed on a trunnion 1102 (shown in FIGS. 68 and 69). The trunnion 1102 is generally cylindrical about the trunnion axis 1101 but tapers from the top surface 1103 of the trunnion 1102 to the bottom surface 1104 of the trunnion 1102. The top and bottom surfaces 1103, 1104 are oriented to be generally orthogonal to the trunnion axis 1101.

Extending from the bottom surface 1104 of the trunnion 1102 is a trunnion neck 1300, which is generally co-axial with the axis 1101 of the trunnion 1102 and generally rectangular when cross-sectioned axially. The trunnion neck 1300 of the femoral component 1000 usually tapers to a larger dimension as it blends into an impacted section 1400 (which includes an impacting axis 1401). Extending from the impacted section 1400, the stem 1200 tapers along a stem axis 1201 to the end 1999 of the femoral component 1000.

As FIGS. 67 and 68 illustrate, the stem axis 1201 is oriented at an angle 1211 relative to the trunnion axis 1101, and, in similar fashion, the impacting axis 1401 is also oriented at an angle 1411 relative to the trunnion axis 1101. Though the foregoing angles 1211, 1411 vary with each manufacturer of femoral components, the angles 1211, 1411 provided in each make and model are well known to surgeons practicing joint replacement. The impacting axis 1401 generally defines the direction in which the femoral component 1000 is inserted into a patient's femur, and those of ordinary skill in the art will understand that the femoral component 1000 is often provided with a cylindrical hole that is axially parallel with the impacting axis 1401 for a tool that impacts the femoral component 1000 into patient's femur.

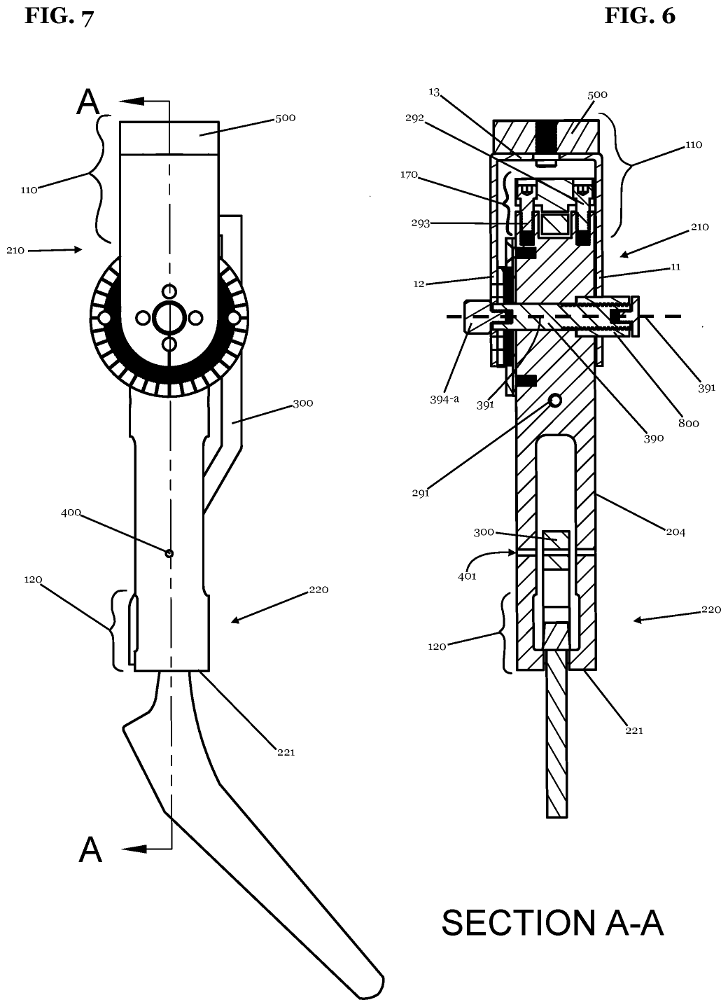

As noted above, the extractor 100 is provided with a body 200, which is shown in FIGS. 2 and 3. As FIGS. 2 and 3 illustrate, the body 200 is provided with an outer body surface 204 that is generally cylindrical about an axis 201. The body 200 is also provided with a first end 210 and a second end 220 (which are also shown in FIGS. 6. and 7). Each of the ends 210, 220 includes an end surface and a portion of the body extending from the end surface. To distinguish the end surfaces from one another, the end surface located at the first end 210 shall be referred to as the “first” end surface 211, and the end surface located at the second end 220 shall be referred to as the “second” end surface 221. As FIG. 2 illustrates, the cylindrically-shaped body 200 extends axially and terminates at each of the end surfaces 211, 221, and each of the end surfaces 211, 221 extends at least partially in a radial direction from the axis 201 and generally terminates at the outer body surface 204.

Located between the first end surface 211 and the second end surface 221, a wedging arrangement 240, a trunnion accepting structure 2102, and a lever opening 230 are defined within the body 200. The wedging arrangement 240 extends axially from the second end surface 221 and is dimensioned so that the neck 1300 of the femoral component 1000 is wedged therewithin. The wedging arrangement 240 is located axially adjacent to the trunnion accepting structure 2102, which is dimensioned to accommodate the trunnion 1102 of the femoral component 1000.

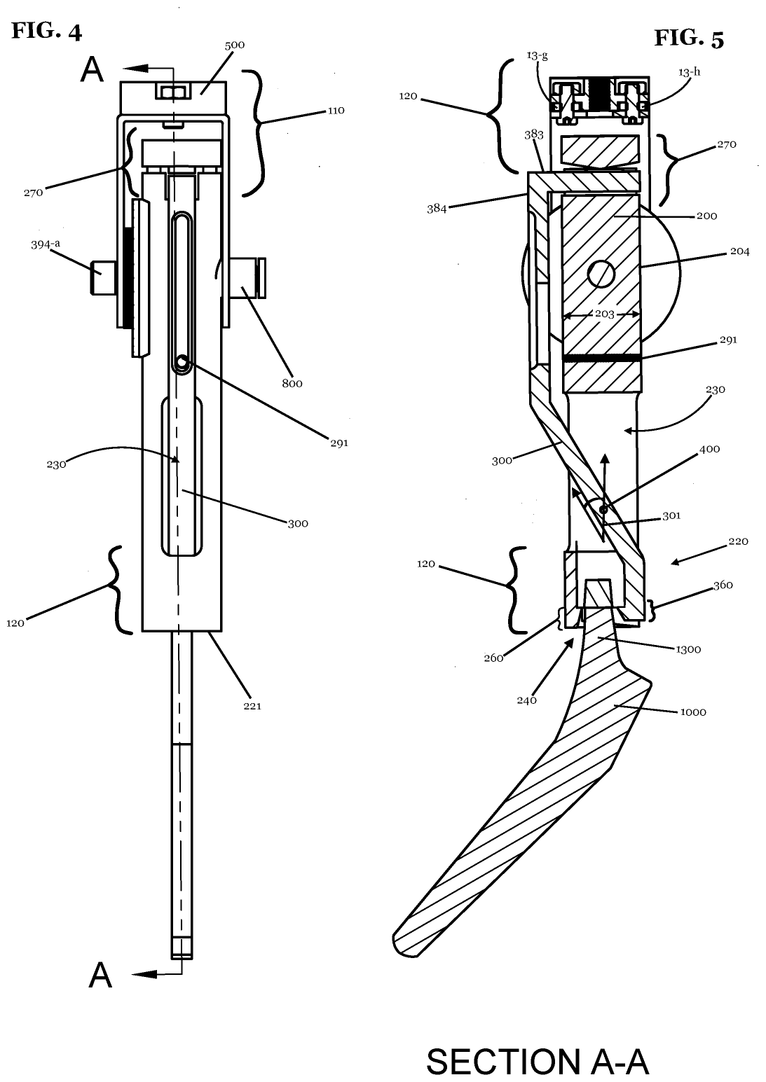

Located adjacent to the trunnion accepting structure 2102 is the lever opening 230. In the preferred embodiment, the lever opening 230 is in the form of a generally rectangular hole extending through the body 200 (though other shapes, such as an ovoid shape, are within the scope of the present invention). As FIGS. 3 and 4 illustrate, the lever opening 230 is shaped to accommodate the lever 300. As FIG. 5 illustrates, the lever 300 extends through the opening 230 at an angle 301 relative to the axis 201 of the body 200; preferably, the angle 301 measures 30 degrees (though it is within the scope of the present invention that the angle 301 measures between 25 and 65 degrees.

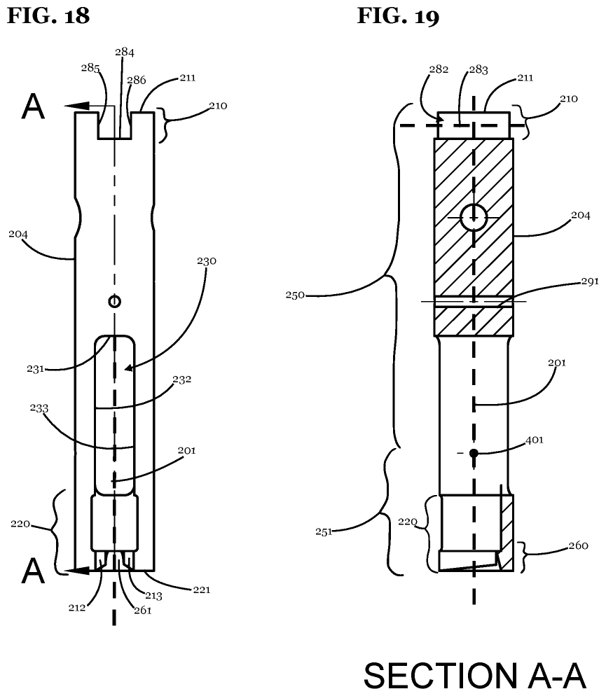

Turning now to FIG. 18, the lever opening 230 is provided with a lever ceiling 231 and two opposing lever walls 232, 233 (designated a “first” lever wall 232 and a “second” lever wall 233 to distinguish one from the other). The lever walls 232, 233 extend from two opposing planes that extend parallel to the axis 201 of the body 200. The lever walls 232, 233 terminate at the lever ceiling 231, which extends between the two opposing lever walls 232, 233 in a plane that is generally orthogonal to the axis 201 of the body 200.

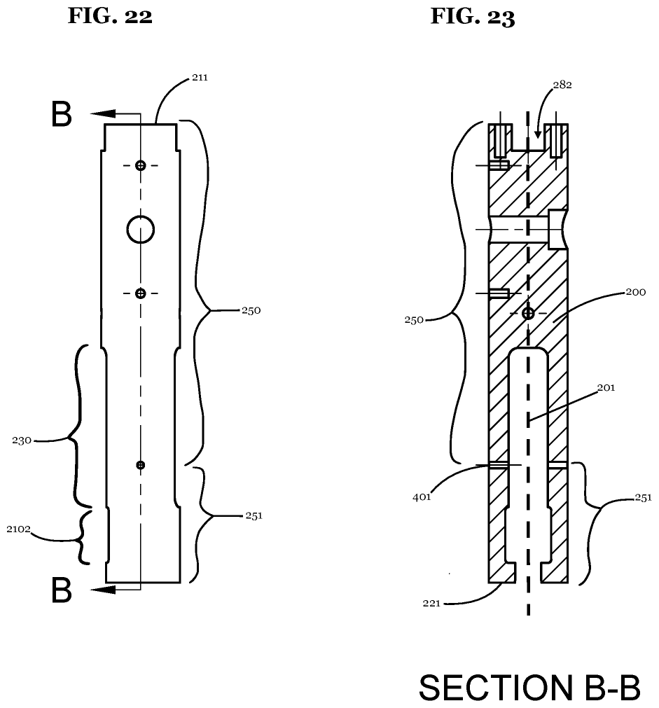

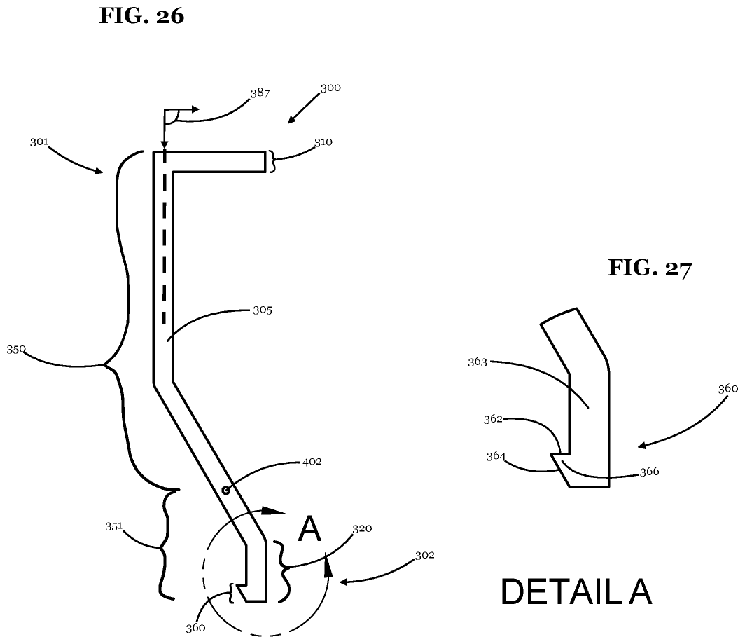

The lever 300 is secured within the opening 230 of the body 200 via the fulcrum 400 (which is in the form of a cylindrically-shaped stainless steel pin extending through an appropriately-sized hole 401 defined within the body 200, as shown in FIG. 6, and an appropriately-sized hole 402 defined within the lever 300, as shown in FIG. 26). As FIGS. 6, 7, 19 and 23 illustrate, the fulcrum hole 401 extends through the body 200 orthogonally relative to the lever 300 and the axis 201 of the body 200. As FIG. 23 also illustrates, the fulcrum hole 401 is located so that it extends through the axis 201 of the body 200 (and hence generally extends through the center of the body 200). Naturally, one of ordinary skill in the art will appreciate that the foregoing positioning of the fulcrum hole 401 is only preferred and that the fulcrum hole 401 can be off-center and still function as a fulcrum 400 within the scope of the present invention.

The body 200 is configured to cooperate with the lever 300, which as shown in FIG. 26, is provided with a first end 310 and a second end 320. The body 200 and the lever 300 are also configured to cooperate with the natural gripping action applied by a surgeon's hand. As noted above, the outer surface 204 of the body 200 is cylindrically-shaped and therefore includes a diameter 203 (which is shown in FIG. 5). The diameter 203 of the body 200 is dimensioned to cooperate with a surgeon's hand; consequently, the diameter 203 of the body 200 measures between (and including) 1 and 3 inches. In the preferred embodiment, the diameter 203 is 1.5 inches.

Because the diameter 203 includes a dimension that ranges between 1 and 3 inches, the surgeon is able to encircle (at least in part) the body 200 with his or her hand and properly grip the extractor 100 while maintaining the surgeon's wrist in general alignment with the forearm (with minimal flexion, extension, or radial or ulnar deviation). Thus, the diameter 203 of the body 200 is dimensioned so that the body 200 and the lever 300 fit within a power gripping arrangement. As used herein, the term “power gripping arrangement” refers to the arrangement of the body 200 and the lever 300 that provide a grip wherein the fingers oppose the position of the thumb while curled about the outer surface 204 of the body 200.

The body 200, the lever 300, and the fulcrum 400 cooperate so that the body 200 and the lever 300 each act as a double Class I lever about the fulcrum 400. Accordingly, the extractor 100 is provided with a first force section 250 and a second force section 350. The extractor 100 is also provided with a first resistance section 251 and a second resistance section 351 (as shown in FIG. 3).

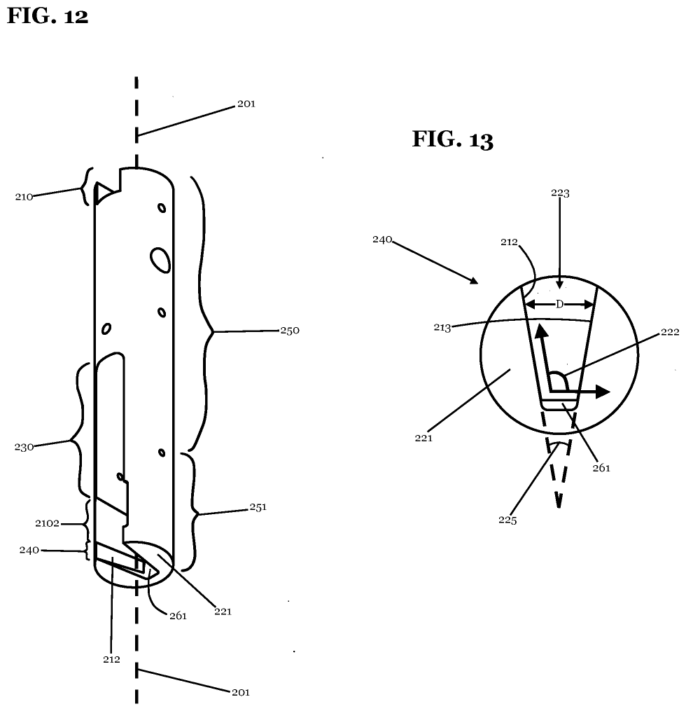

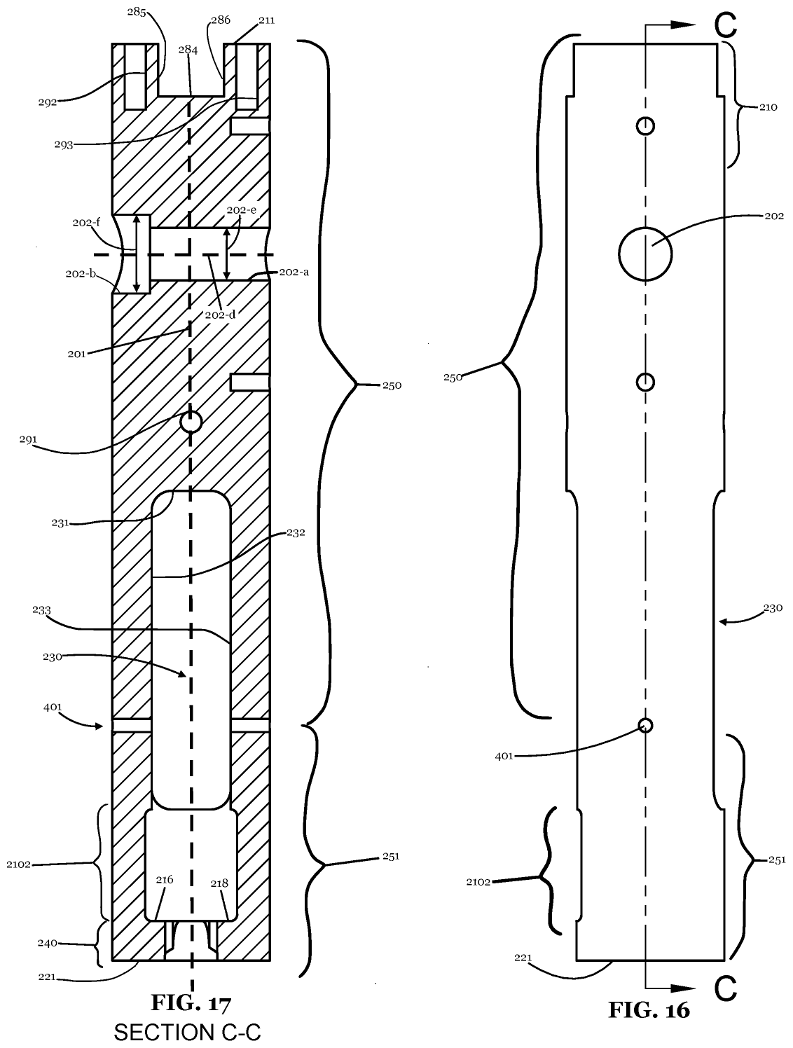

The first force section 250 and the first resistance section 251 are located on the body 200 (as FIGS. 12, 16, 17, 19, 22, and 23 illustrate) while the lever 300 provides the extractor 100 with the second force section 350 and the second resistance section 351 are located on the lever 300 (as FIG. 26 illustrates). As shown in FIGS. 16, 17, 19, and 23, the first force section 250 extends from the fulcrum hole 401 toward the first end 210 of the body 200 while the first resistance section 251 extends from the fulcrum hole 401 to the second end 220 of the body 200. Similarly, as FIG. 26 illustrates, the second force section 350 extends from the fulcrum hole 402 toward the first end 310 of the lever 300 while the second resistance section 351 extends from the fulcrum hole 402 toward the second end 320 of the lever 300. Each of the force sections 250, 350 is positioned so that each opposes the other while rotating about the fulcrum 400 (as FIG. 3 depicts).

As FIG. 3 further depicts, the extractor 100 is provided with a clamping arrangement 130, which is located at the second end 120 of the extractor 100. Referring now to FIG. 5, the clamping arrangement 130 includes opposing clamping sections, a clamping section 260 located on the body 200 and a clamping section 360 located on the lever 300. (To distinguish the clamping section 360 of the lever 300 from the clamping section 260 of the body 200, the clamping section 260 of the body 200 shall be referred to as the “first” clamping section 260 while the clamping section 360 of the lever 300 shall be referred to as the “second” section 360 or “second clamping” section 360.)

The first clamping section 260 is located at the second end 220 of the body 200 while the second clamping section 360 is located at the second end 320 of the lever 300. As FIG. 5 illustrates, the first and second clamping sections 260, 360 cooperate with each other to clamp the neck 1300 of the femoral component 1000. The first and second clamping section 260, 360 also cooperate with the gripping force of a surgeon's hand to clamp the neck 1300 of the femoral component 1000.

With the force sections 250, 350 acting about the fulcrum 400, the extractor 100 cooperates with the gripping force of the surgeon's hand to clamp the neck 1300 of the femoral component 1000 at the second end 220 of the body 200. The mechanical advantage obtained from the first and second force sections 250, 350 is further increased by the positioning of the fulcrum 400 towards the second end 220 of the body 200. As FIGS. 3 and 5 illustrate, the fulcrum 400 is positioned towards the second end 220 to provide the extractor 100 with increased force sections 250, 350.

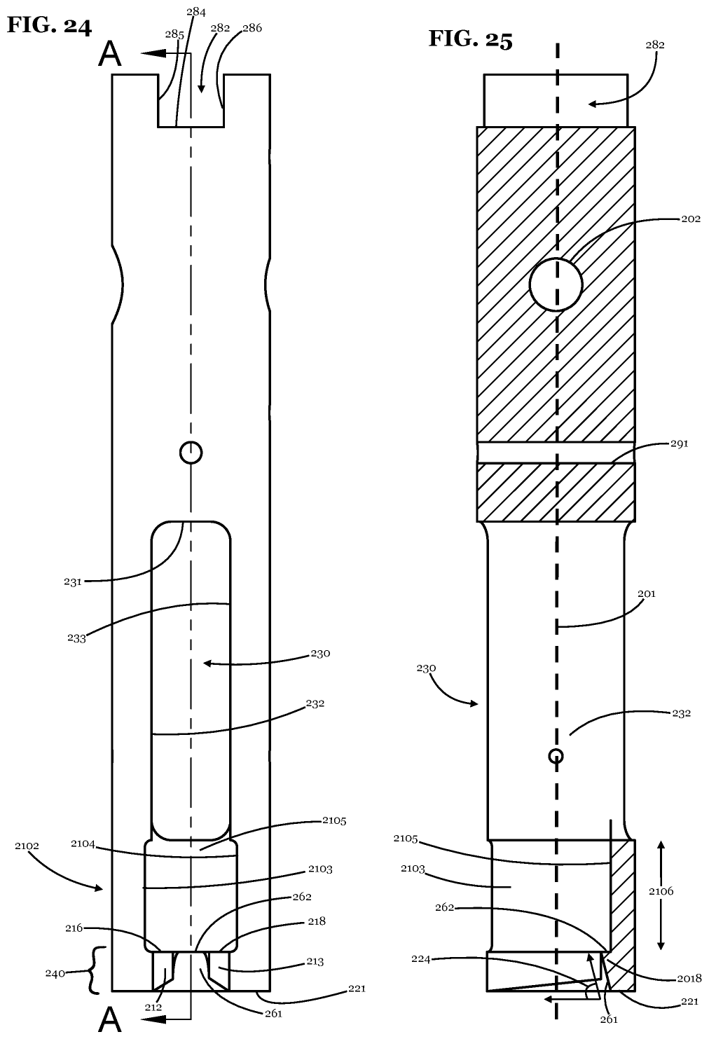

Turning now to FIGS. 24 and 25, the extractor 100 is provided with an upper clamping surface 262 located on the clamping section 260 of the body 200 (which is shown in FIG. 5). The extractor 100 is also provided with an upper clamping surface 362 located on the lever 300 (shown in FIGS. 26 and 27). To distinguish one from the other, the upper clamping surface 262 located on the clamping section 260 of the body 200 shall be referred to as the “first” upper clamping surface 262 while the upper clamping surface 362 located on the lever 300 shall be referred to as the “second” upper clamping surface 362.

The first upper clamping surface 262 of the body 200 is oriented to be generally parallel with the second end surface 221 and generally orthogonal relative to the axis 201 of the body 200. The first upper clamping surface 262 abuts a first clamping surface 261, which, in turn, extends axially from the second end surface 221 and preferably at an angle 224 relative to the axis 201 of the body 200. As FIGS. 3 and 5 illustrate, the angle 224 measures 75 degrees; however, in alternative embodiments, the angle 224 ranges between 45 and 60 degrees. The first upper clamping surface 262 is generally co-planar with upper wedging surfaces 216, 218 (which are themselves co-planar with each other), and, in such a configuration, the first upper clamping surface 262 and the co-planar upper wedging surfaces 216, 218 form a single plane and thus a single surface which is oriented to be generally orthogonal to the axis 201 of the body 200.

Referring now to FIGS. 24 and 25, the upper wedging surfaces 216, 218 partially define the trunnion accepting structure 2102, which is located axially within the body 200 between the upper wedging surfaces 216, 218 and the lever opening 230. The trunnion accepting structure 2102 is provided with a plurality of trunnion accepting walls 2103, 2104, 2105 (referred to as “first,” “second,” and “third” trunnion accepting walls respectively). The first trunnion accepting wall 2103 is orthogonal to the first upper wedging surface 216 and extends axially from the first upper wedging surface 216 to the lever opening 230. The first trunnion accepting wall 2103 is generally parallel to the axis 201 of the body 200 and the first lever wall 232.

A second trunnion accepting wall 2104 is orthogonal to the second wedging surface 218 and extends axially from the second upper wedging surface 218 to the lever opening 230. Similar to the first trunnion accepting wall 2103, the second trunnion accepting wall 2103 is generally parallel to the axis 201 of the body 200 and the second lever wall 233. The first and second trunnion accepting walls 2103, 2104 are generally parallel to each other and spaced apart from each other so that the trunnion 1102 of a femoral component 1000 can fit between the two walls 2103, 2104.

Abutting the first and second trunnion accepting walls 2103, 2104 is a third trunnion accepting wall 2105. The third trunnion accepting wall 2105 is orthogonal to the first upper clamping surface 262 and extends axially from the from the first upper clamping surface 262 and terminates at the lever opening 230. The third trunnion accepting wall 2105 is oriented to be generally orthogonal to the first and second trunnion accepting walls 2103, 2104 and thus provides spacing between the first and second trunnion accepting walls 2103, 2104. In the preferred embodiment, the third trunnion accepting wall is at least 14 mm in width so that the first and second trunnion accepting walls 2103, 2104 are at least 14 mm apart. The preferred width of the third trunnion accepting wall 2105 is 22.222 mm so that the first and second trunnion accepting walls 2103, 2104 are spaced 22.222 mm apart.

The trunnion accepting walls 2103, 2104 extend from lever walls 232, 233 (with the first trunnion accepting wall 2103 extending from the first lever wall 232 and the second trunnion accepting wall 2104 extending from the second lever wall 233). As noted above, the first and second trunnion accepting walls 2103, 2104 terminate at the wedging structure 240 where the upper wedging surfaces 216, 218 are located.

Referring now to FIGS. 12, 14, and 18, the wedging structure 240 is located at the second end 220 of the body 200 (and the second end 120 of the extractor 100). As noted above, the wedging structure 240 is configured to wedge the second end 220 of the body 200 around the neck 1300 of the femoral component 1000 (at least partially). The wedging structure 240 is provided with a first wedging surface 212, a second wedging surface 213, a first upper wedging surface 216 and a second supper wedging surface 218. The wedging surfaces 212, 213 extend axially from the second end surface 221 and, in the preferred embodiment, abut the upper wedging surfaces 216, 218.

Sandwiched between the wedging surfaces 212, 213 is a first clamping surface 261, as FIGS. 12, 13, and 18 illustrate. Each of the wedging surfaces 212, 213 extends from the first clamping surface 261 so that an angle 222 is formed relative to the plane of the first clamping surface 261. As shown in FIG. 13, the angle 222 in the preferred embodiment measures 100 degrees; however, in an alternative embodiment, the angle measures between (and including) 90 and 110 degrees. Because each of the wedging surfaces 212, 213 extends from the first clamping surface 261 at an angle 222, the wedging surfaces are spaced from each other a distance (designated “D” in FIG. 13) that varies between 0.2 and 0.85 inches, with the preferred range being between 0.2 and 0.75 inches.

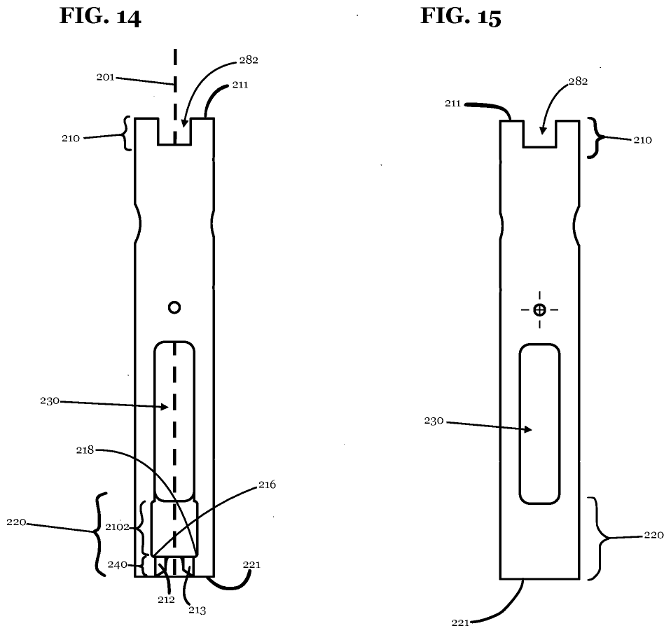

As FIGS. 14, 17, and 24 illustrate, the wedging structure 240 includes the upper wedging surfaces 216, 218 which extend from the wedging surfaces 212, 213 in a generally orthogonal orientation. Because each of the wedging surfaces 212, 213 is oriented at an angle 222 and because the upper wedging surfaces 216, 218 extend from the wedging surfaces 212, 214, a spacing is created between the upper wedging surfaces 216, 218 that varies as the spacing between the wedging surfaces 212, 213 varies. Accordingly, the upper wedging surfaces 216, 218 are spaced from each other a distance “D” that ranges between 0.2 and 0.85 inches, with the preferred range being between 0.2 and 0.75 inches.

Because at least one of the wedging surfaces 212, 213 (preferably both) is oriented at an angle 222, the wedging surfaces 212, 213 provide the second end surface 221 of the body 200 with a tapered opening 223, as FIG. 13 illustrates. As noted above, the wedging surfaces 212, 213 abut the first clamping surface 261 located within the first clamping section 260. As FIG. 25 illustrates, the first clamping surface 261 is oriented to extend from the second end surface 221 at an angle 224 relative to the plane of the end surface 221 that measures 75 degrees. Though the angle 224 of the preferred embodiment measures 75 degrees, one of ordinary skill in the art will appreciate that the angle 224 in alternative embodiments ranges between 45 and 90 degrees. The first clamping surface 261 extends axially from the second end surface 221 and abuts the upper clamping surface 262 to form a tooth (which is designated “2018”).

As noted above, the extractor 100 is provided with a lever 300. In the preferred embodiment, the lever 300 is an integral bar of stainless steel with a first end 301 and a second end 302 that has been bent to form a plurality of sections. In the preferred embodiment, the lever 300 is rectangular in cross-sectional shape; however, in alternative embodiments, the lever 300 is circular or hexagonal in shape and formed by welding various sections together.

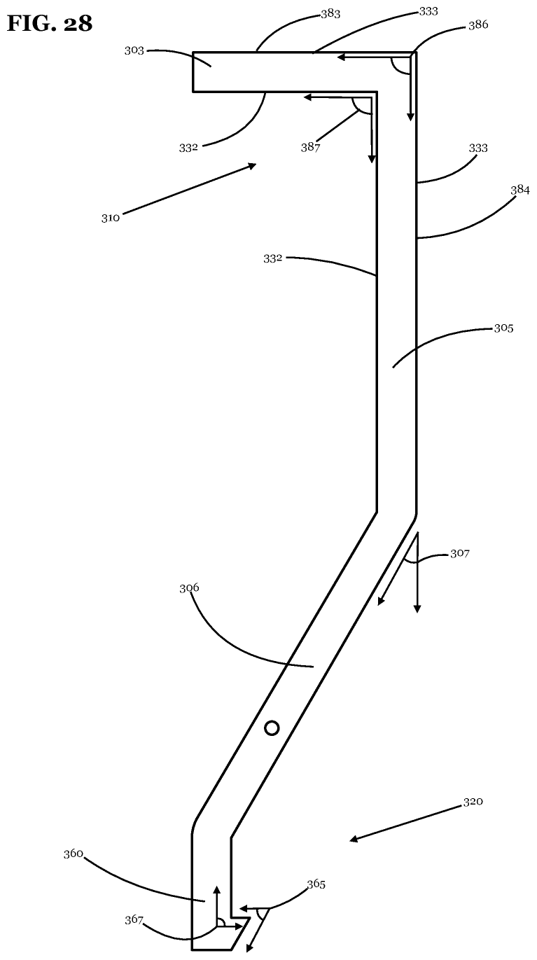

As FIG. 28 discloses, the lever 300 includes a first section 303, which extends from the first lever end 310, and a second section 360, which extends from the second lever end 320. (As noted above, the second section 360 is also referred to as the “second clamping section 360.”) The lever 300 also includes a third section 305 and a fourth section 306. What has been identified as a “first” section 303 (and what shall also be referred to as a “locking” section 303) is provided with an orthogonal lever locking surface 383. The first section 303 (or locking section 303) abuts the third section 305 of the lever 300 to form an angle 387 that measures between 85 and 100 degrees. The third section 305 abuts the fourth section 306, which abuts the second section 360 (or the second clamping section 360).

The third section 305 of the lever 300 is located within the force section 350 of the lever 300 and is oriented to be generally parallel to the second section 360. As the foregoing implies, the third section 305 and the second section 360 (or second clamping section 360) are joined via the fourth section 306. Thus, the fourth section 306 extends between the third and second sections 305, 360 at an angle 307 that preferably measures 30 degrees, but, in alternative embodiments, ranges between 25 and 35 degrees. Consequently, the fourth section 306 is also referred to herein as the “angled” section 306 of the lever 300.

Extending from the fourth (or “angled”) section 306 of the lever 300, the second section 360 (also referred to herein as the clamping section 360) functions as a clamp upon the neck 1300 of the femoral component 1000. As FIGS. 26 and 27 illustrate, the clamping section 360 of the lever 300 is provided with an upper lever clamping surface 362, and a trunnion clearance section 363. The trunnion clearance section 363 extends from the angled section 306 of the lever 300 and terminates at the upper lever clamping surface 362. As FIG. 28 illustrates, the upper lever clamping surface 362 extends from the trunnion clearance section 363 at an angle 367 that, in the preferred embodiment, measures 90 degrees. However, in an alternative embodiment, the upper lever clamping surface 362 is a curved surface that forms a plurality of angles with the trunnion clearance section 363 (in which case, the angle 367 measures between 0 and 90 degrees).

The upper lever clamping surface 362 terminates where it meets a lower lever clamping surface 364. The upper lever clamping surface 362 and the lower lever clamping surface 364 form an angle 365 that measures less than 90 degrees (though it is within the scope of the present invention for the angle to measure 90 degrees). In the preferred embodiment, the angle 365 measures 60 degrees. As FIG. 27 illustrates, the upper lever clamping surface 362 abuts the lower lever clamping surface 364 to form a tooth ridge that functions as a tooth (and, as a result, the tooth ridge thus formed shall be referred to simply as a “tooth 366”).

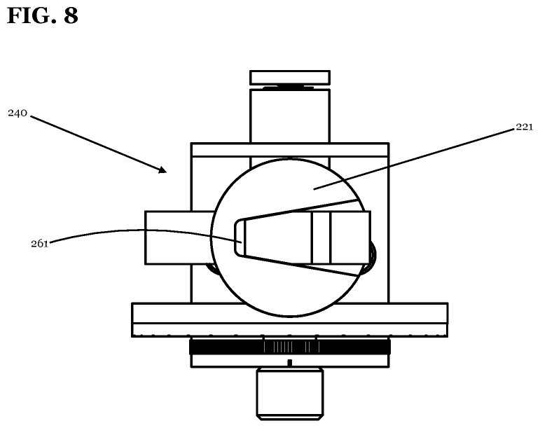

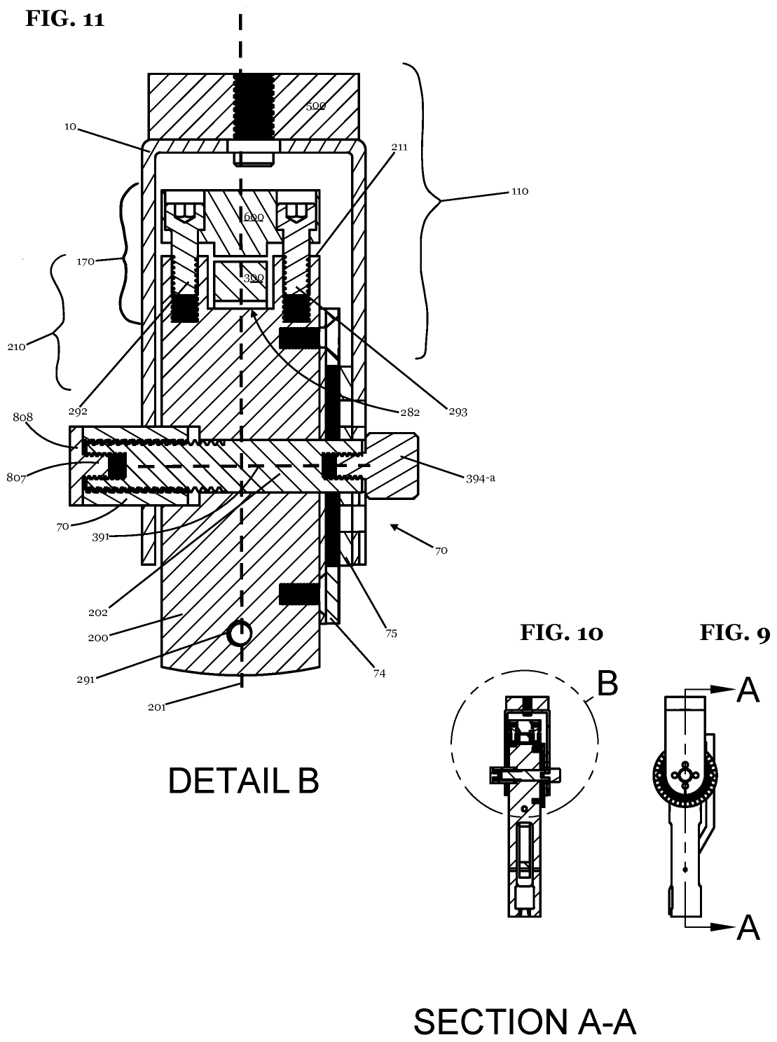

Referring now to FIGS. 9-11, various views of the extractor 100 are shown. FIG. 10 is a cross-sectional view of the extractor 100 shown in FIG. 9, and FIG. 11 is a detailed cross-sectional view of the extractor 100 shown in FIG. 10. The foregoing figures depict the first end 210 of the body 200 configured to cooperate with the lever 300, (while FIGS. 5 and 8 depict the second end 220 of the body 200 configured to wedge the clamping arrangement 230 under the trunnion 1102 and onto the neck 1300 of the femoral component 1000).

As FIGS. 11 and 19 illustrate, a first threaded surface 291 is defined within the force section 250 of body 200. The first threaded surface 291 is coarsely threaded, preferably with a ¼-20 UNC thread profile, though a finely threaded profile (such as ¼-28 UNF) may also be used. Though a coarse female thread is preferred, in an alternative embodiment, the body 200 is provided with a first threaded surface 291 that is in the form of a male threaded stud that is press-fit into an appropriately dimensioned hole within the body 200 (such as a stud with ¼-20 UNC or ¼-28 UNF thread profile).

As FIG. 19 shows, the first threaded surface 291 extends through the body 200 and is oriented to extend orthogonally through the axis 201 of the body 200. Though the first threaded surface 291 is oriented to be orthogonal to the axis 201, in an alternative embodiment, the first threaded surface 291 is oriented at an angle that measures less than 90 degree relative to the axis 201, such as 45 degrees, or greater than 90 degrees relative to the axis 201, such as 135 degrees. Though the first threaded surface 291 extends through the axis 201 of the body 200 in the preferred embodiment, in alternative embodiments, the first threaded surface 291 extends though the body 200 offset from the axis 201.

The first end 110 of the extractor 100 is provided with a fastener (preferably a plurality of fasteners) that are configured to lock the lever 300 into the first end 210 of the body 200 after the clamping arrangement 130 has been secured to the neck 1300 of the femoral component 1000. Accordingly, the first end 110 of the extractor 100 is provided with a locking arrangement 170, and, as FIG. 11 depicts, the locking arrangement 170 is provided with a threaded surface and a fastener. In the preferred embodiment, the locking arrangement 170 is provided with a plurality of threaded surfaces (referred to as a “second” threaded surface 292 and a “third” threaded surface 293 to distinguish one from the other).

As FIG. 11 illustrates, the threaded surfaces 292, 293 are defined within the body 200 and extend axially from the first end surface 211 towards the second end surface 221. The threaded surfaces 292, 293 are dimensioned according to a plurality of male threaded fasteners. In the preferred embodiment, the second and third threaded surfaces 292, 293 are blind holes that have been drilled on a bolt circle measuring 1 1/16 in diameter so that each of the threaded surfaces 292, 293 is located 180 degrees from the other and positioned on either side of a slot 282 defined within the first end 210 of the body 200 (as shown in FIGS. 14, 15, and 23-25). It is preferred that the second and third threaded surfaces 292, 293 are coarsely threaded with a ¼-20 UNC thread profile; however, in an alternative embodiment, the threaded surfaces 292, 293 are finely threaded with a ¼-28 UNF thread profile.

While the preferred embodiment is provided with threaded surfaces 292, 293 that accept male threaded fasteners, such as a socket head cap screw, in an alternative embodiment, the threaded surfaces 292, 293 accept female threaded fasteners such as nuts. In such an alternative embodiment, the threaded surfaces 292, 293 extend axially from the first end surface 211 in the form of threaded studs that have been press-fit into holes extending into the body 200.

Returning again to FIG. 11, the locking arrangement 170 includes a slot 282, which is defined within the first end 210 of the body 200. The slot 282 is both positioned and dimensioned according to the lever 300. In the preferred embodiment, the slot 282 is positioned on the body 200 so that it is in alignment with the lever 300. The slot 282 is dimensioned to accept at least a portion of the lever 300 therewithin as FIG. 11 depicts. Thus, the slot 282 extends both axially into the body 200 (as FIGS. 14 and 15 illustrate) and radially across the first end surface 211 (as FIGS. 19 and 23 illustrate). As FIGS. 19 and 23 further illustrate, the slot 282 is generally rectangular in cross-section and is provided with a slot axis 283 that is generally orthogonal to the axis 201 of the body 200. As FIGS. 17, 18, and 24 illustrate, the slot 282 is defined by a slot floor 284 that forms a plane that is generally orthogonal to the slot axis 283; the slot 212 is further defined by two opposing slot walls (designated a “first” slot wall 285 and a “second” slot wall 286 to distinguish one from the other) that extend orthogonally from the slot floor 284 with each terminating at the first end surface 211 of the body 200.

As FIGS. 17, 18, and 24 make clear, the plane of the slot floor 284 and the plane of the lever ceiling 231 are generally parallel to each other. Similarly, each of the slot walls 285, 286 forms a plane that is generally parallel to the plane of at least one of the lever walls 232, 233. In the preferred embodiment, the plane of the first slot wall 215 is generally parallel to the plane of the first lever wall 232; in the same vein, the plane of the second slot wall 286 is generally parallel to the plane of the second lever wall 233. Thus, the slot 282 and the lever opening 230 are oriented to extend through the body 200 so that each is generally parallel to the other.

As noted above, and as shown in FIG. 11, the slot 282 is shaped to accept at least a portion of the first end 310 of the lever 300. The lever 300 is provided with an angle 387 (shown in FIG. 26) formed from the locking section 303 and the third section 305 of the lever 300. The angle 387 is dimensioned so that the locking section 303 (shown in FIG. 28) extends into the slot 282 defined within the first end 210 of the body 200 when the extractor 100 is being clamped onto the femoral component 1000. As noted above, and as FIG. 28 further shows, the locking section 303 of the lever 300 extends from the first lever end 310 and abuts the third section 305 of the lever 300 so that angle 387 measures between 85 and 100 degrees.

As FIG. 28 additionally illustrates, the lever 300 is provided with a surface that is internal to the angle 387 between the locking section 303 and the third section 305 as well as a surface that is external to the angle 387 between the locking section 303 and the third section 305. The surface that is internal to the angle 387 between the locking section 303 and the third section 305 shall be referred herein to as an “interior lever surface 332” while the surface that is external to the angle 330 between the locking section 303 and the third section 305 shall be referred to herein as an “exterior lever surface 333.” The exterior lever surface 333 includes the orthogonal lever locking surface 383 (which extends from the extreme end of the locking section 303 and terminates at the third section 305 of the lever 300). Extending from where the orthogonal lever locking surface 383 terminates, a parallel lever locking surface 384, located on the third section 305 within the force section 350 of the extractor 100, extends to the fourth (or “angled”) section 306 of the lever 300.

Turning now to FIG. 3, the orthogonal lever locking surface 383 is oriented to be generally orthogonal to the axis 201 of the body 200 when the lever 300 is clamped onto the neck 1300 of the femoral component 1000 and locked in place. In contrast, the parallel lever locking surface 384 is oriented to be generally parallel relative to the axis 201 of the body 200 when the lever 300 is clamped onto the neck 1300 of the femoral component 1000. Thus, the orthogonal lever locking surface 383 forms an angle 386 with the parallel lever locking surface 384 that measures between 85 and 100 degrees with the preferred angle 386 measuring 95 degrees. To fabricate the angle 386 between the orthogonal lever locking surface 383 and the parallel lever locking surface 384, rectangular bar stock is bent so that the locking section 303 is generally perpendicular relative to the third section 305; then the orthogonal lever locking surface 383 is machined so that the preferred angle 386 with the parallel lever locking surface 384 is achieved.

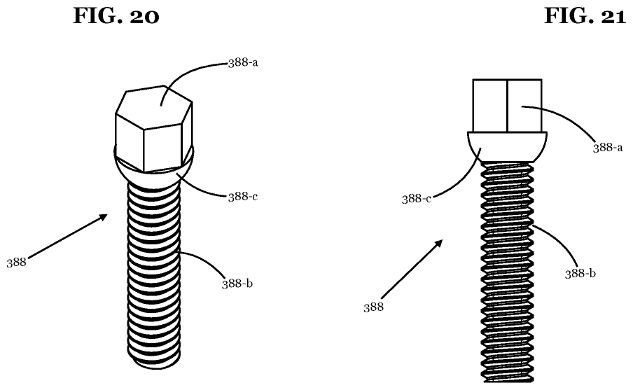

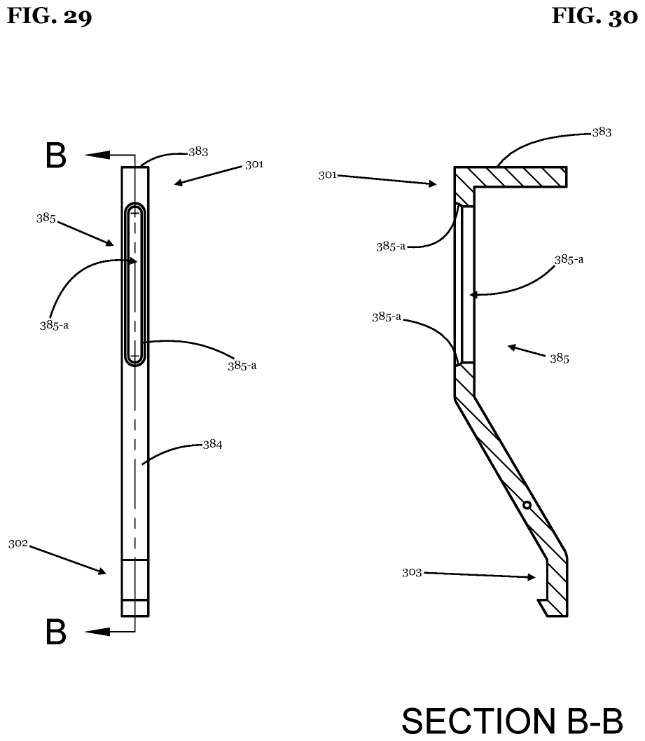

The parallel lever locking surface 384 is provided with a grooved slot 385 that is shaped to cooperate with a male threaded fastener 388 (shown in FIGS. 20-21) with a head 388-a and a shank 388-b, preferably a bolt that is provided with a hexagonal head and a ¼-20 UNC threaded shank. For ease of reference, the male threaded fastener 388 shall be referred simply as the “first fastener 388” in order to distinguish this fastener from other fasteners referred to herein. Referring now to FIGS. 29 and 30, the grooved slot 385 includes a groove 385-a that is semi-circular in cross-section and a slot 385-b that is defined within the groove 385-a. The groove is dimensioned so that the head 388-b of the first fastener 388 clamps the third section 305 of the lever 300 while the slot 385-b is dimensioned so that the shank 388-b passes through. Advantageously, the bearing surface 388-c of the first fastener 388 is spherically shaped to fit within the groove 385-a. Alternatively, a spherically or semi-spherically shaped washer could also be employed.

To clamp the extractor 200 onto the neck 1300 of the femoral component 1000, the lever 300 is rotated about the fulcrum 400 so that the clamping section 360 is moved to a position in closer proximity to the clamping section 260 of the body 200. Thus, the angle 386 between the orthogonal lever locking surface 383 and the third section 305 of the lever 300 is dimensioned according to the slot 282 defined within the first end 210 of the body 200. The orthogonal lever locking surface 383 is oriented so that it is generally orthogonal relative to the third section 305 of the lever 300. Consequently, the angle 386 measures between 85 and 100 degrees with the preferred range being between 90 and 95 degrees. In the preferred embodiment, the angle 386 is created by bending rectangular bar stock and then machining the orthogonal lever locking surface 383.

When the lever 300 is locked into place onto the body 200, the second section 304 (and the third section 305, which is parallel to the second section 304) are oriented relative to the body 200 so as to form an angle relative to the axis 201 measuring between +5 degrees and −5 degrees, with the preferred range being +5 degrees and −3 degrees.

As noted above, when the extractor 100 is being clamped onto the neck 1300 of the femoral component 1000, at least a portion of the lever 300 extends through the lever opening 230. As is also noted above, the lever 300 is rotatably secured to the body 200 via the fulcrum 400 so that the clamping section 360 of the lever 300 rotates towards the clamping section 260 of the body 200. Accordingly, when the clamping section 360 of the lever 300 is rotated towards the clamping section 260 of the body 200, the locking section 303 of the lever 300 is simultaneously rotated towards the locking arrangement 270 located at the first end 110 of the extractor 100. Conversely, when the extractor 100 releases the neck 1300 of the femoral component 1000, the clamping section 360 of the lever 300 is rotated away from the clamping section 260 of the body 200, and the locking section 303 of the lever 300 is simultaneously rotated away from the locking arrangement 270 located at the first end 110 of the extractor 100.

As the locking section 303 of the lever 300 is rotated towards the locking arrangement 270 located at the first end 110 of the extractor 100, the locking section 303 of the lever 300 enters the slot 282 at the first end 210 of the body 200 with the orthogonal lever locking surface 383 facing away from the slot floor 284. At the same time, the clamping section 360 of the lever 300 approaches the wedging structure 240 located within the clamping section 260 of the body 200. Consequently, the clamping section 360 of the lever 300 approaches the wedging structure 240, which is located within the clamping section 260 of the body 200 (and hence approaches the wedging surfaces 212, 213 which are located within the wedging structure 240). Thus, as the lever 300 pivots about the fulcrum 400, at least a portion of the lever 300 is accommodated within the slot 282, which is located at the first end 210 of the body 200, and, at least a portion of the lever 300 is accommodated within the wedging arrangement 240, which is located at the second end 220 of the body 200.

As noted above, the body 200 and the lever 300 are dimensioned to provide a power gripping arrangement; by squeezing the body 200 and the third section 304 of the lever 300 together, the surgeon rotates the clamping section 360 of the lever 300 and the clamping section 260 of the body 200 closer together. As is also noted above, in the foregoing power gripping arrangement, the body 200 itself and the lever 300 together act as a class I lever. Additionally, as is further noted above, the fulcrum 400 is positioned towards the clamping arrangement 130 of the extractor 100, thereby increasing the force sections 250, 350 of the extractor 100 and decreasing the resistance sections 251, 351 of the extractor 100.

With the force sections 250, 350 increased and the resistance sections 251, 351 decreased, the mechanical advantage of the extractor 100 is increased. Thus, the gripping force applied to the force sections 250, 350 of the extractor 100 yields a greater clamping force at the resistance sections 251, 351 where the clamping arrangement 130 of the extractor 100 is located.

The clamping force of the clamping arrangement 130 is further increased by the fastening structures included with the extractor 100. As FIGS. 2, 4, 5, 17, 19, and 25 illustrate, the body 200 is provided with a threaded surface, preferably a plurality of threaded surfaces in a plurality of orientations. As noted above, the body 200 is provided with a first threaded surface 291 that extends into the body 200 in an orientation that is generally orthogonal to the axis 201 of the body 200. The body 200 is also provided with second and third threaded surfaces 292, 293 that extend into the body 200 in an orientation that is generally parallel to the axis 201 of the body 200.

Like the first threaded surface 291, the second and third threaded surfaces 292, 293 are coarsely threaded, preferably with a ¼-20 UNC thread profile defined within the body 200, though a finely threaded profile (such as ¼-28 UNF) may also be used. Though a female thread is preferred, in an alternative embodiment, the body 200 is provided with second and third threaded surfaces 292, 293 that are in the form of a male threaded stud that is press-fit into an appropriately dimensioned hole within the body 200 (such as a stud with ¼-20 UNC or ¼-28 UNF thread profile).

As noted above, the second and third threaded surfaces 292, 293 are oriented to be parallel to the axis 201 of the body 200. Though the second and third threaded surfaces 292, 293 are oriented to be parallel to the axis 201, in an alternative embodiment, the second and third threaded surfaces 292, 293 are oriented at an angle that measures less than 180 degrees relative to the axis 201, such as 135 degrees.

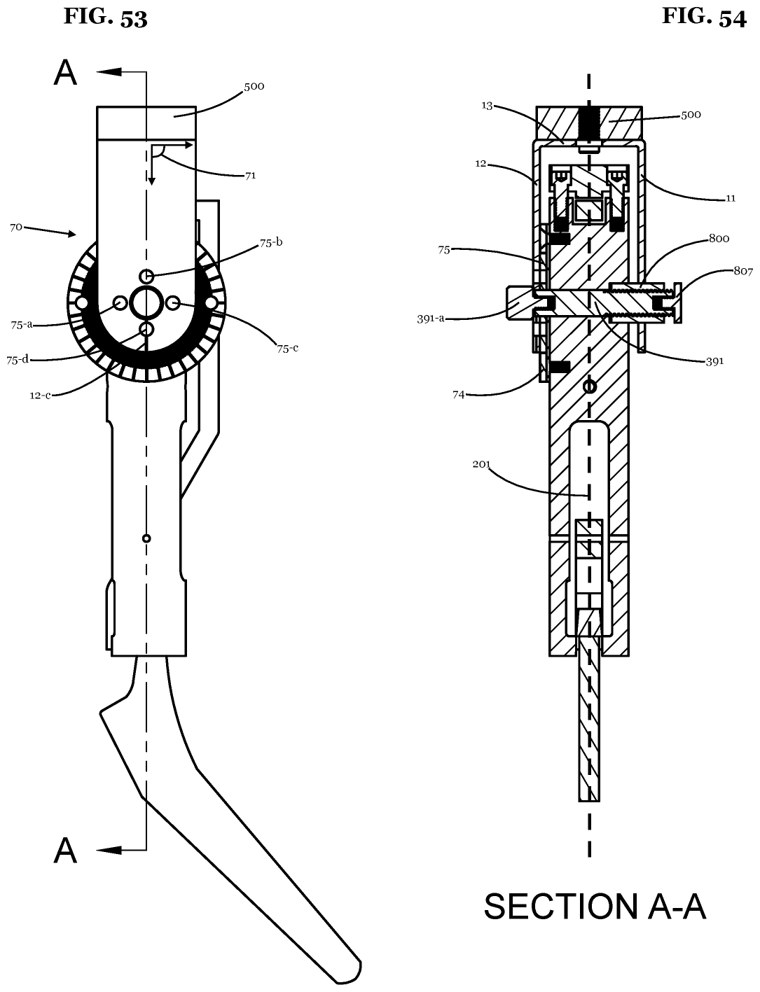

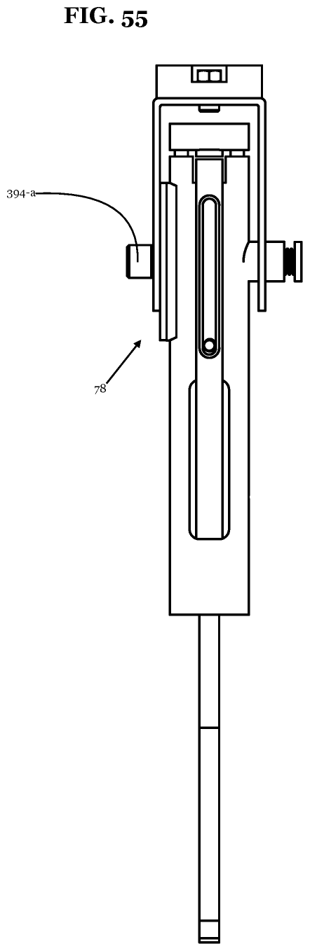

Referring now to FIGS. 53 and 54, the preferred embodiment is shown clamped onto the neck 1300 of the femoral component 1000 and locked in place. To clamp the extractor 100 onto the neck 1300 of the femoral component 1000, the lever 300 is rotated so that the first end 310 rotates away from the first end 210 of the body 200 (and hence towards the second end 220). Because the lever 300 moves about the fulcrum 400, the second end 320 of the lever 300 is also rotated, albeit in the opposite direction (namely, away from the second end 220 of the body 200 and towards the first end 210 of the body 200). Thus, the second end 320 of the lever 300 is rotated away from the trunnion accepting structure 2102. The trunnion accepting structure 2102 is then placed over the trunnion 1102 of the femoral component 1000 so that at least a portion of the upper wedging surfaces 216, 218 of the body 200 is in contact with the bottom surface 1104 of the trunnion 1102. By placing the upper wedging surfaces 216, 218 into contact with the bottom surface 1104 of the trunnion 1102, the upper wedging surfaces 216, 218 of the body 200 are oriented to be generally parallel with the bottom surface 1104 of the trunnion 1102. Because the bottom surface 1104 of the trunnion 1102 is generally orthogonal to the trunnion axis 1101 and because the upper wedging surfaces 216, 218 are generally orthogonal to the axis 201 of the body 200, the axis 201 of the body 200 is generally parallel with the axis 1101 of the trunnion 1102.

After the trunnion 1102 is placed within the trunnion accepting structure 2102, the lever 300 is rotated so that the first end 310 approaches the first end 210 of the body 200 (and, accordingly, the second end 320 of the lever 300 approaches the second end 220 of the body 200). As described above, the lever 300 and the body 200 are configured to clamp the neck 1300 of the femoral component 1000 and wedge the trunnion 1102 within the trunnion accepting structure 2102. As is further described above, the clamping section 360 of the lever 300, as well as the clamping section 260 of the body 200, are each provided with a tooth 366, 2018. The lever 300 is dimensioned to rotate about the fulcrum 400 so that the tooth 366 on the clamping section 360 of the lever 300 opposes the tooth 2018 on the clamping section 260 of the body 200. Thus, the opposing teeth 366, 2018 form a set of clamping jaws. (Consequently, the teeth 366, 2018 shall also be referred to herein as “clamping jaws” 366, 2018.)

With the upper wedging surfaces 216, 218 of the body 200 contacting the bottom surface 1104 of the trunnion 1102, the tooth 366 on the clamping section 360 of the lever 300 pushes the neck 1300 of the femoral component 1000 toward the opposing tooth 2018 on the clamping section 260 of the body 200 when the third section 304 of the lever 300 is pressed towards the body 200. Thus, when the force sections 250, 350 of the extractor 100 are pressed together, the resistance sections 251, 351 clamp the neck 1300 of the femoral component 1000.

As the tooth 366 on the clamping section 360 of the lever 300 pushes the neck 1300 toward the opposing tooth 2018, the neck 1300 is pushed into the wedging surfaces 212, 213 of the wedging arrangement 240 and at least a portion of the bottom surface 1104 of the trunnion 1102 is positioned axially so that it is generally parallel to a portion of at least one of the upper wedging surfaces 216, 218. With the bottom surface 1104 positioned axially over a portion of at least one of the upper wedging surfaces 216, 218, at least one of the upper wedging surfaces 216, 218 exerts a normal force upon at least a portion of the bottom surface 1104 of the trunnion 1102 when the extractor 100 is impacted. Naturally, it is preferred that the bottom surface 1104 of the trunnion 1102 be positioned axially over both of the upper wedging surfaces 216, 218 so that both upper wedging surfaces 216, 218 exert a normal force upon the bottom surface 1104 of the trunnion 1102 when the extractor 1000 is impacted.

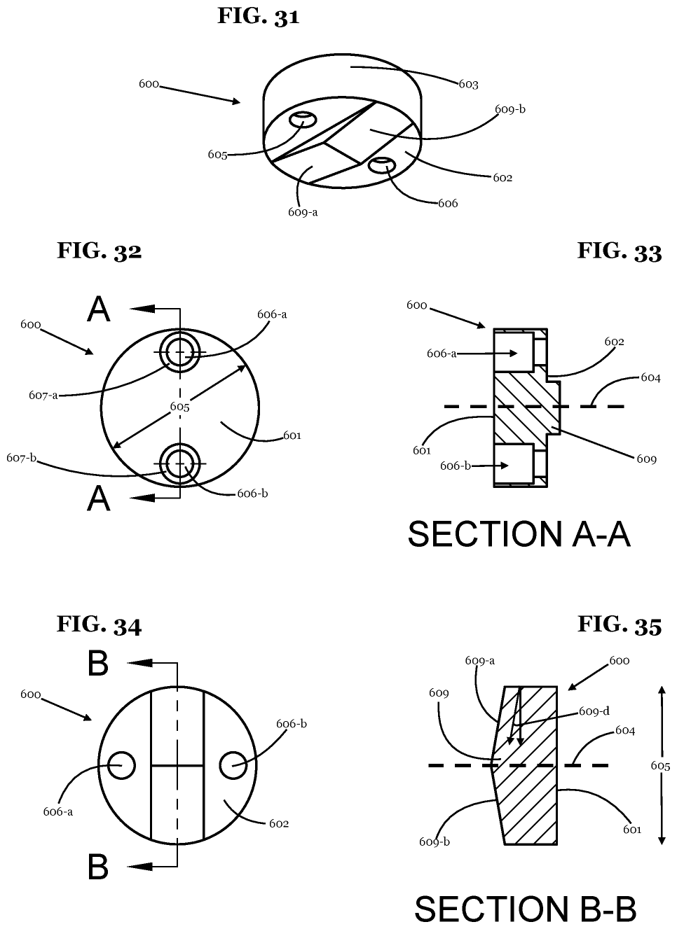

To prevent the clamping sections 260, 360 from loosening, the extractor 100 is provided with a locking component 600. As FIGS. 31-35 illustrate, the locking component 600 is shaped according to the body 200 and includes a plurality of surfaces and structures. The locking component 600 is provided with a first outer surface 601, a second outer surface 602, and a third outer surface 603. The third outer surface 603 is cylindrical about an axis 604 and provided with a diameter 605 that is dimensioned according to the diameter 203 of the body 200. In the preferred embodiment, both diameters 203, 605 are 1.5 inches. The cylindrically-shaped third outer surface 603 abuts the first and second outer surfaces 601, 602, which generally extend radially from the axis 604.

A through-hole extends from the first outer surface 601 to the second outer surface 602. As FIGS. 31-35 illustrate, the preferred embodiment is provided with a plurality of through-holes 606-a, 606-b that are circular and dimensioned to accept a pin or a male fastener. The second outer surface 602 is provided with a locking ridge 609 that is formed where two ramps 609-a, 609-b abut one another. Each of the ramps 609-a, 609-b extends radially from the axis 604 to the third outer surface 603. Each of the ramps 609-a, 609-b also extends axially from the second outer surface 602 as each ramp extends radially inward from the third outer surface 603 to form a crest 609-c where each ramp meets the other.

In the preferred embodiment, the through-holes 606-a, 606-b have been drilled on a bolt circle extending around the axis 604 that matches have been drilled on a bolt circle measuring 1 1/16 in diameter so that each of the through-holes 606-a, 606-b is located 180 degrees from the other and positioned on either side of the ramps 609-a, 609-b. The through-holes 606-a, 606-b are then counter-bored to accommodate male threaded fasteners (which, in the preferred embodiment, are ¼-20 UNC socket head cap screws). For ease of reference, the foregoing male threaded fasteners shall be referred to as “socket head cap screws.”

As the foregoing indicates, the through-holes 606-a, 606-b are positioned to line up with the threaded surfaces 291, 292 of the body 200, and the ramps 609-a, 609-b and the locking ridge 609 are positioned to extend within the slot 282 of the body 200. Thus, when the socket head cap screws are passed through the through-holes 606-a, 606-b and torqued into the threaded surfaces 291, 292 of the body 200, the ramps 609-a, 609-b and the locking ridge 609 extend into the slot 282 of the body 200.

As noted above, the fulcrum 400 is positioned so that the mechanical advantage of the first and second force sections 250, 350 of the extractor 100 is increased. As a result, when the surgeon squeezes the first and second force sections 250, 350 together, the tooth 366 on the lever 300 exerts a greater force upon the neck 1300 of the trunnion 1102 thereby forcing the neck 1300 into the wedging surfaces 212, 213 of the body 200 thereby creating an interference fit between the second end 120 of the extractor 100 and the neck 1300 of the femoral component 1000. Much as the upper wedging surfaces 212, 213 exert a normal force upon the bottom trunnion surface 1104, the upper lever clamping surface 362 on the lever 300 contacts the bottom trunnion surface 1104 and exerts a normal force upon the bottom trunnion surface 362 when the extractor 100 is impacted.

After the lever 300 and the body 200 are positioned so that the extractor 100 is in clamping and wedging engagement with the femoral component 1000, the lever 300 and the body 200 are locked in place. By torqueing the first fastener 388 into the first threaded surface 291 defined within the body 200, the head 388-a of the first fastener 388 clamps the parallel lever locking surface 384 of the lever 300 and draws the third section 305 of the lever 300 towards the body 200. Because the first threaded surface 291 is located in the first force section 250 of the extractor 100 and the parallel locking surface 384 of the third section 305 of the lever 300 is located in the second force section 350 of the extractor 100, the clamping force exerted by the first fastener 388 exerts a greater clamping force upon the jaws 366, 2018 clamping the femoral component 1000.

The locking component 600 further locks in place the lever 300 and the body 200. As noted above, when the neck 1300 of the femoral component 1000 is clamped, the locking section 303 of the lever 300 is disposed (at least partially) within the slot 282 defined at the first end 210 of the body 200. Furthermore, when the locking section 303 of the lever 300 is disposed within the slot 282 of the body 200, the orthogonal lever locking surface 383 faces away from the slot floor 284 and therefore faces the locking ridge 609 of the locking component 600. As a result, when the socket head cap screws are passed through the holes 606-a, 606-b defined within the locking component 600 and torqued into the threaded surfaces 291, 292 of the body 200, the locking ridge 609 is pressed into the orthogonal lever locking surface 383 of the lever 300. Thus, the socket head cap screws lock the lever 300 and the body 200 in clamping and wedging engagement with the femoral component 1000.

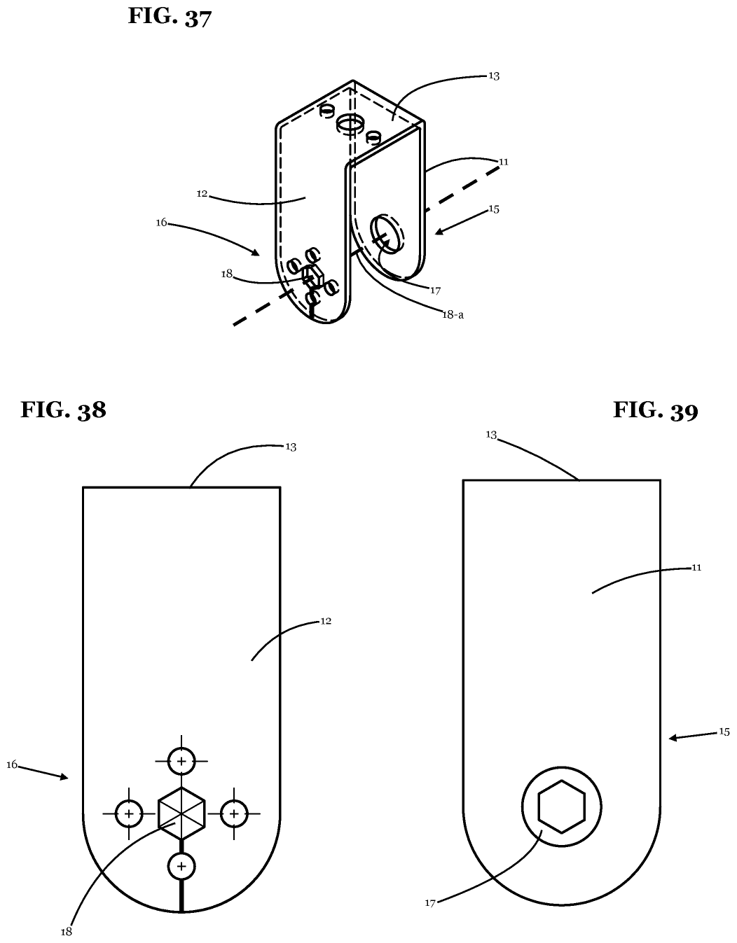

As FIG. 11 illustrates, the extractor 100 is provided with a bracket 10, which rotates on a shaft 390 that includes an axis 391 extending through the body 200. The bracket 10 is configured to cooperate with the angle selector 70 and therefore is provided with a plurality of sections (as FIGS. 37-41 illustrate). By stamping or bending a piece of ⅛ inch stainless steel plate, the bracket 10 is provided with a leg section and a plate section. Ideally, the bracket is provided with a plurality of leg sections; accordingly, as FIGS. 37-41 illustrate, the preferred embodiment is provided with a first leg section 11 and a second leg section 12. The leg sections 11, 12 of the bracket 10 extend from a plate section 13 at an angle 14 that measure 90 degrees; however, in alternative embodiments, the angle 14 measures between (and including) 85 and 95 degrees.

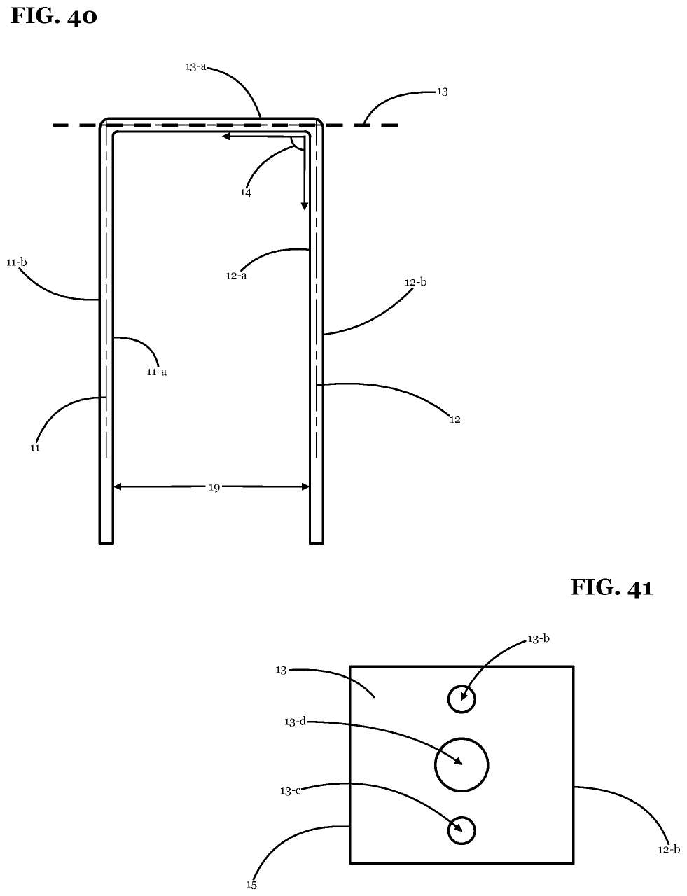

As FIG. 40 illustrates, the bracket 10 is provided with a “U” shape with the plate section 13 located between the leg sections 11, 12. According to one aspect, the leg sections 11, 12 are dimensioned, at least in part, according to the body 200. According to another aspect, the leg sections 11, 12 are dimensioned to provide clearance for the plate section 13 to rotate about the first end 210 of the body 200.

Each of the leg sections 11, 12 is provided with an inner leg surface and an outer leg surface. Thus, the first leg section 11 is provided with a “first” inner leg surface 11-a and a “first” outer leg surface 11-b while the second leg section 12 is provided with a “second” inner leg surface 12-a and a “second” outer leg surface 12-b. (Consistent with the ordinal number convention used herein, the “first” and “second” monikers are used simply to distinguish one surface from another.

The first and second inner surfaces 11-a, 12-a extend from the plate section 13 of the bracket 10 generally parallel to each other. Located within the “U” shape of the bracket 10, the first and second inner surface 11-a, 12-a face one another. In contrast, the first and second outer leg surfaces 11-b, 12-b are located outside the “U” shape of the bracket 10 and face away from one another.

Each of the leg sections 11, 12 extends from the plate section 13 and terminates at leg ends 15, 16. (As used herein, the term “leg end” is to be understood broadly to include both the extreme end, as well as a portion of the leg section adjacent to the extreme end.) FIGS. 37-41 illustrate an opening defined within each of the leg ends 15, 16. Accordingly, the first leg end 15 is provided with a first leg opening 17 that is circular in shape and dimensioned according to a tightener 800. The second leg end 16 is provided with a second leg opening 18 that is out-of-round in shape (preferably hexagonal) and dimensioned to transmit torque between the bracket 10 and the shaft 390.

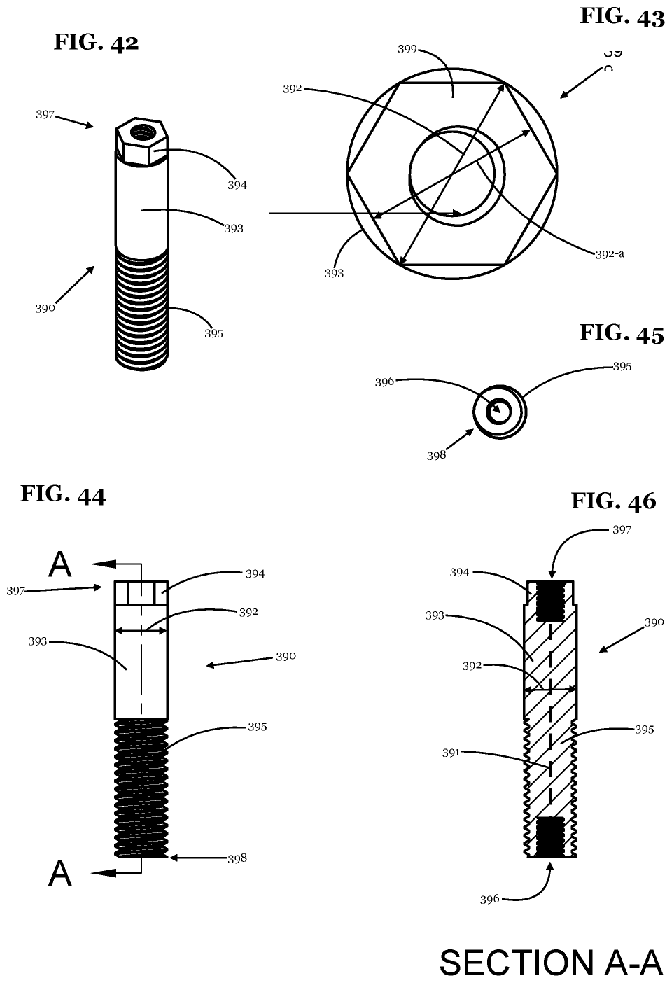

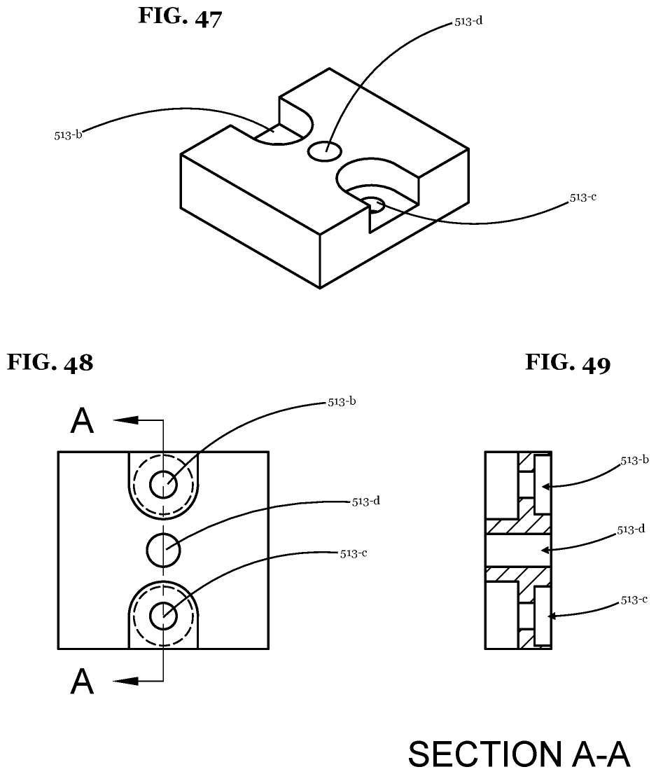

As the foregoing implies, the second leg opening 18 is shaped according to the shaft 390, which is slidably and rotatably secured within the body 200 of the extractor 100. As FIGS. 42-46 illustrate, the shaft 390 is provided with a shaft axis 391, an out-of-round shaft section 394, and a threaded shaft section 395. Though the foregoing may imply that the shaft 390 is composed of a plurality of sections that are assembled together (and it is within the scope of the present invention that the shaft be fabricated in such a manner), an assembly of discrete shaft sections is not preferred. Rather, it is preferred to manufacture the shaft 390 from round stainless steel rod that has been cut to length, turned to a circular diameter, and then milled and threaded to shape.

Accordingly, the shaft 390 constituting the preferred embodiment includes a cylindrical shaft section 393 that has been turned to a diameter 392 measuring ½ inches. The shaft diameter 392 is dimensioned to provide the shaft 390 and the body 200 with a close fit wherein the shaft 390 rotates and slides axially within the body 200. The cylindrical shaft section 393 and at least a portion of the threaded shaft section 395 are disposed within the body 200 so that the shaft axis 391 extends through the axis 201 of the body 200 in a generally orthogonal orientation. The cylindrical shaft section 393 is located between the out-of-round shaft section 394 and the threaded shaft section 395. Thus, the out-of-round shaft section 394 and the threaded shaft section 395 extend axially from the cylindrical shaft section 393 to extreme the ends 398, 399 of the shaft 390 itself.

As noted above, the out-of-round shaft section 394 extends axially from the cylindrical shaft section 393 and terminates to provide one of the extreme ends 398, 399 to the shaft 390; thus, the extreme end 399 of the shaft 390 is provided with an out-of-round cross-sectional shape, preferably an out-of-round cross-sectional shape that is hexagonal. As is also noted above, the out-of-round shaft section 394 is shaped so that torque applied to the out-of-round shaft section 394 is transmitted to the shaft 390. As FIGS. 43 and 39 illustrate, the out-of-round shaft section 394 is also shaped to fit within the second leg opening 18 so that torque applied to the bracket 10 is transmitted to the shaft 390 (and torque applied to the shaft 390 is transmitted to the bracket 10). As a result, the out-of-round shaft section 394 closely fits within the out-of-round shape of the second leg opening 18.

In contrast to the out-of-round shaft section 394, the threaded shaft section 395 is generally circular in cross-sectional shape, and therefore, the extreme end 398 of the shaft 390 is circular in cross-sectional shape. (To distinguish each of the extreme ends 398, 399 from the other, the extreme end 398 that terminates the threaded shaft section 395 shall also be referred to as the “circular extreme end 398” while the other extreme end 398 that terminates the out-of-round shaft section 394 shall also be referred to as the “out-of-round extreme end 399.”)

A blind hole is defined within the shaft 390 and extends from an extreme end. In the preferred embodiment, the shaft 390 is provided with a plurality of shaft holes 396, 397 defined within the extreme ends 398, 399. To distinguish each of the shaft holes 396, 397 from the other, the shaft hole 396 extending from the circular extreme end 398 shall be referred to as the “first shaft hole 396” while the other shaft hole 397, which extends from the out-of-round extreme end 399, shall be referred to as the “second shaft hole 397.” In the preferred embodiment, the shaft holes 396, 397 extend axially into the shaft 390 in alignment with the shaft axis 391 and are threaded to receive male threaded fasteners. However, in an alternative embodiment, each of the shaft holes 396, 397 is unthreaded to receive a fastening pin.

As noted above, the bracket 10 rotates on the shaft 390 and thus is provided with an axis 18-a of rotation (as shown in FIG. 37). The leg openings 17, 18 are defined within the ends 15, 16 of the legs 11, 12 so that they are co-axial with each other and the axis 18-a of rotation of the bracket 10. The plate section 13 forms a plane (designated “13-a”) and is dimensioned so that the leg sections 11, 12 are generally parallel to each other and spaced apart a predetermined distance (referred to herein as a “spacing distance” and designated “19” in FIG. 40). The spacing distance 19 extends linearly along the plane 13-a of the plate section 13 in a direction that is parallel to the axis 391 of the shaft 390. Because the axis 391 of the shaft 390 is oriented to be orthogonal relative to the axis 201 of the body 200, the plane 13-a of the plate section 13 is also oriented to be orthogonal relative to the axis 201 of the body 200.

The spacing distance 19 is dimensioned so that the bracket 10 and the shaft 390 move in line with the axis 391 and orthogonally relative to the axis 201 of the body 200. Defined within the plate section 13 is a hole, preferably a plurality of holes 13-b, 13-c, 13-d, configured to cooperate with an attachment. The holes 13-b, 13-c, 13-d are configured to cooperate with a fastener. In FIG. 3, the holes 13-b, 13-c, 13-d are shown cooperating with a fastener; as shown therein, the holes 13-b, 13-c are shown cooperating with a plurality of threaded fasteners 13-e, 13-f. The fasteners 13-e, 13-f are male threaded fasteners, which are secured to the plate section 13 by corresponding female threaded fasteners 13-g, 13-h (shown in cross-section in FIG. 5) in the form of hex nuts (though in an alternative embodiment, the fasteners 13-e, 13-f are secured to the plate section by welding). The fasteners 13-e, 13-f are evenly spaced about a bolt circle and provide the extractor 100 with means for securing an attachment, which in the preferred embodiment is a strike plate 500.

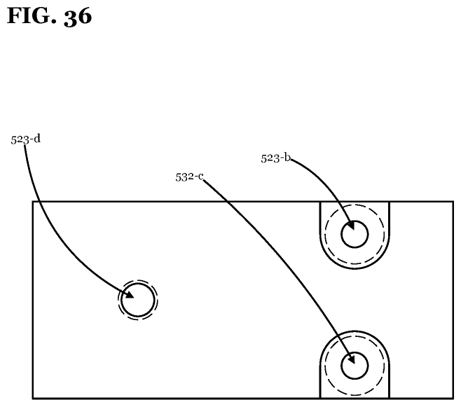

The bracket 10 is also provided with a hole 13-d defined within the center of the plate section 13 (and hence shall be referred to as the “center hole” 13-d to distinguish it from the other holes 13-b, 13-c in the plate section 13). The center hole 13-d is threaded (and hence circular in shape) and generally co-axial with the axis 201 of the body 200. Thus, the center hole 13-d provides the extractor 100 with means for fastening a male threaded attachment. In the preferred embodiment, the male threaded attachment is a slap hammer (not shown), a Whelan extractor strike plate (not shown) or a strike plate that has been offset from the axis 201 of the body 200 (shown in FIG. 36).

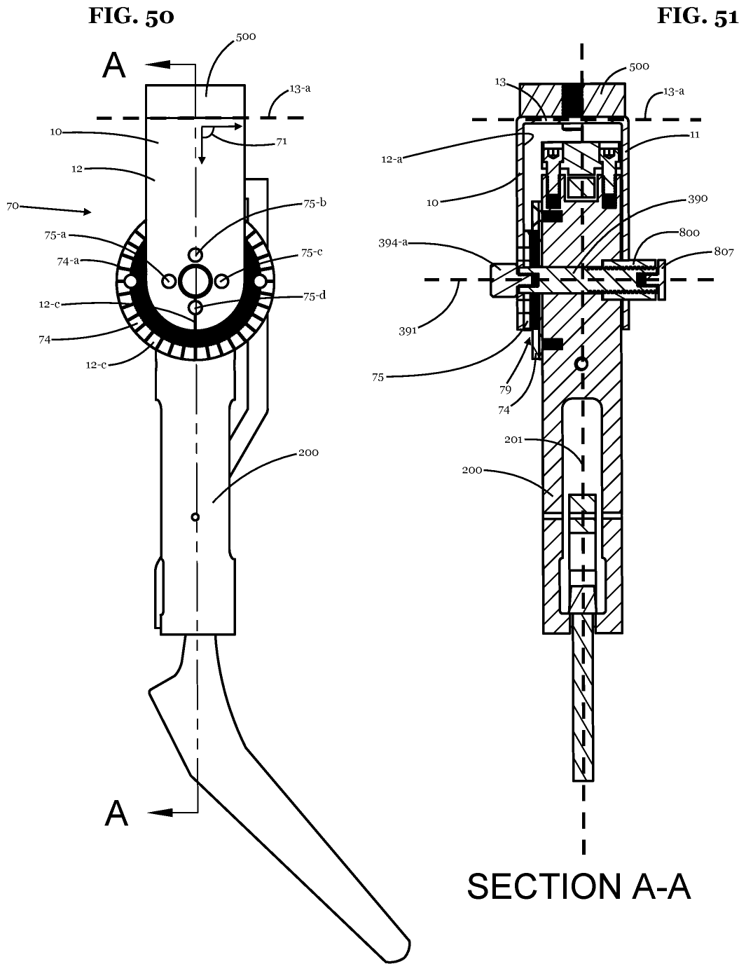

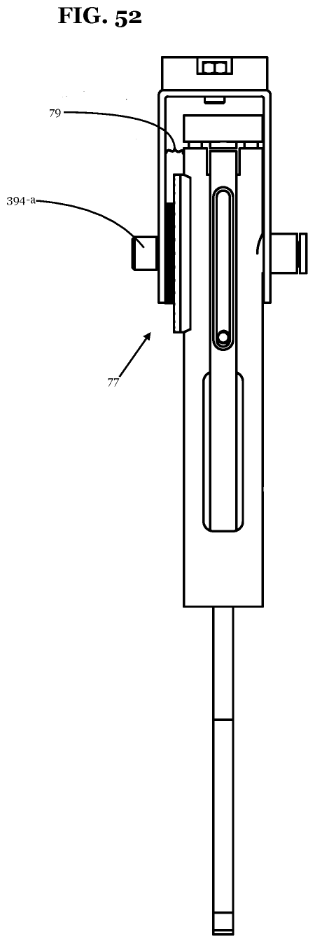

As noted above, the extractor 100 is provided with an angle selector 70. The angle selector 70 is configured to rotate about the axis 391 of the shaft 390 and lock into a predetermined angle between the plane 13-a of the plate section 13 of the bracket 10 and the axis 201 of the body 200. Thus, the angle selector 70 moves between two configurations a rotating configuration 77 (depicted in FIGS. 50-52) and a locking configuration 78 (depicted in FIGS. 53-55). In the preferred embodiment, the angle selector 70 is provided with a “locking distance,” a dimension designated “79” in FIGS. 51 and 52 that separates the rotating configuration 77 from the locking configuration 78.

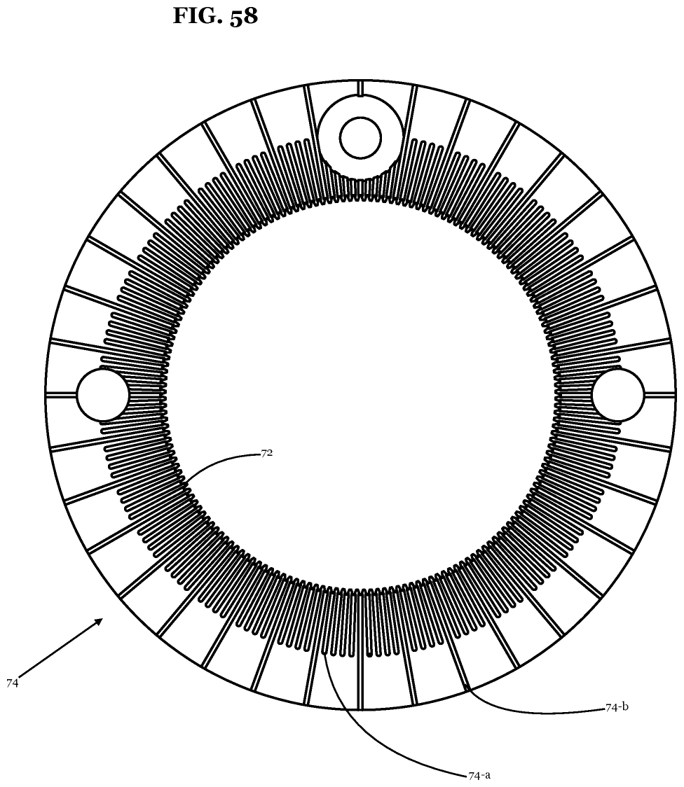

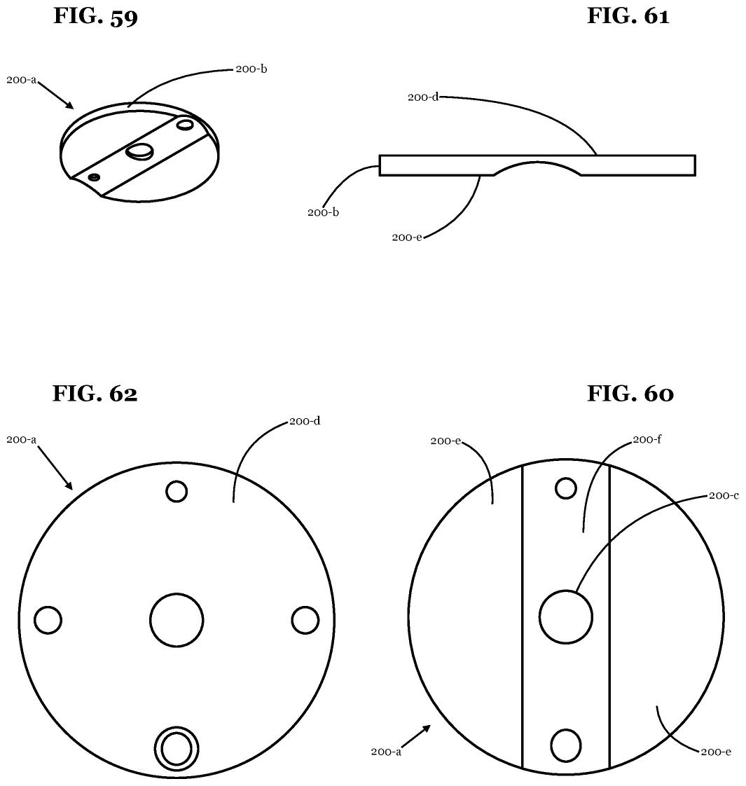

The angle selector 70 is also provided with a plurality of angle settings 72. In the preferred embodiment, the angle settings 72 are in the form of teeth on interlocking gears 74, 75 (as is shown in FIGS. 57 and 58). However, in an alternative embodiment, the angle settings 72 are in the form of splines on interlocking internal and external splines. In yet another alternative embodiment, the angle settings 72 are in the form of sides on a polygonal shaft that fits within a correspondingly-shaped polygonal opening formed within the body 200.

In the preferred embodiment, the angle selector 70 is illustrated in FIG. 11. As shown therein, the angle selector 70 is provided with a first gear 74 and a second gear. The first and second gears 74, 75 are each provided with teeth that mesh with the teeth on the other. The first gear 74 is secured to the body 200 while the second gear 75 is secured to the bracket 10. The first gear 74 is in the form of a ring gear that is secured to the body 200 in axial alignment with a hole 202 defined within the body 200 and a tooth in axial alignment with the axis 201 of the body 200. The hole 202 is circular in shape and extends orthogonally through the axis 201 of the body 200.

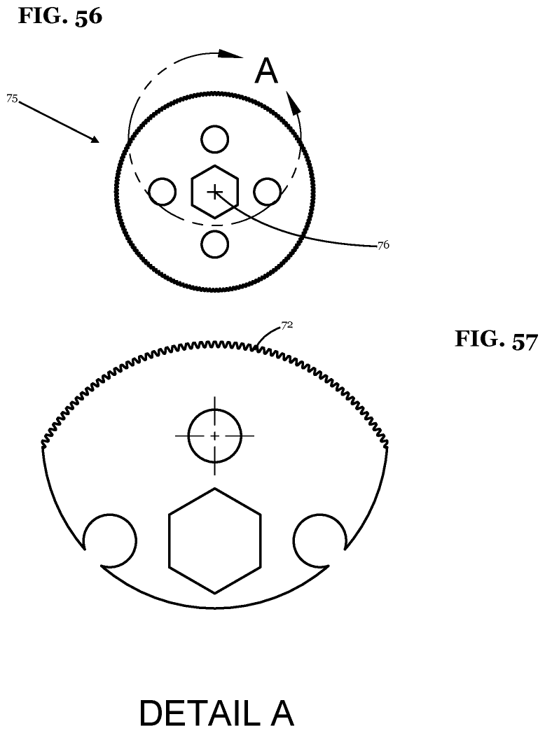

The second gear 75 is in the form of a spur gear that is provided with an axis 76 of rotation. The second gear 75 is attached to the second inner leg surface 12-b of the second leg 12 of the bracket 10 so that the axis 76 of the second gear 75 (shown as “+” in FIG. 56) is in alignment with the axis 18-a of the second leg opening 18 (for ease of reference, the foregoing attachment of the second gear 75 and the bracket 10 shall be referred to as a “bracket-gear subassembly”). In the preferred embodiment, the second gear 75 is attached to the second inner leg surface 12-b via a plurality of stainless steel pins 75-a, 75-b, 75-c, 75-d. After the bracket-gear subassembly is completed, the bracket-gear subassembly is assembled onto the out-of-round shaft section 394 of the shaft 390.

In the preferred embodiment, the out-of-round shaft section 394 of the shaft 390 is hexagonal in shape and closely fits within the hexagonal shape of the second leg opening 18. The out-of-round shaft section 394 of the shaft 390 and the out-of-round second leg opening 18 are dimensioned so that each is provided with a diameter that is less than the diameter 392 of the cylindrical shaft section 393. As one of ordinary skill in the art will appreciate, the shape of a hexagon is provided with at least two diameters: a diameter extending through the center between opposing sides (as shown in FIG. 43 and designated “392-a”) and a diameter extending through the center between opposing vertices (as shown in FIG. 43 and designated “392”). As one of ordinary skill in the art will also appreciate, the diameter between opposing sides of a hexagon is less than the diameter between opposing vertices.

Because the diameter 392 of the cylindrical shaft section 393 is larger than a diameter 392-a of the out-of-round leg opening 18, the larger diameter 392 of the cylindrical shaft section 393 acts as a stop for the axial positioning of the bracket-gear subassembly on the shaft 390. After the bracket-gear subassembly is positioned onto the out-of-round section 394 of the shaft 390, a thumb screw fastener 394-a (shown in FIG. 67) is torqued into the second shaft hole 397 extending axially into the shaft 390 from the out-of-round extreme end 399 of the shaft 390. Thus, the bracket-gear subassembly is firmly secured to the shaft 390.

The bracket-gear subassembly, the shaft 390, and the body 200 are secured so that the body 200 is positioned between the inner leg surface 11-a, 12-a and the shaft 390 extends through the body 200. Because the first gear 74 is attached to the body 200, the first gear 74 is also positioned between the inner leg surfaces 11-a, 12-a. With the second gear 75 secured to the second inner leg surface 12-a, the body 200 with the first gear 74 attached thereto is positioned between the inner leg surfaces 11-a, 12-a so that the first gear 74 faces the second gear 75 and the gears 74, 75 are in axial alignment with each other.

With the gears 74, 75 placed in axial alignment, the axis 18-a of the leg openings 17, 18 is aligned with the axis of the circular hole 202 extending through the body 200. After being thus positioned in axial alignment, the shaft 390 is placed through the hole 202 defined within the body 200 and secured to the bracket-gear subassembly. Thus, when the extractor 100 is fully assembled, the gears 74, 75, the shaft 390, and the leg openings 17, 18 defined within the legs 11, 12 of the bracket 10 are all in axial alignment.

As the foregoing indicates, the hole 202 extending through the body 200 is dimensioned at least in part according to the shaft 390. In the preferred embodiment, and as FIG. 17 illustrates, the hole 202 is generally cylindrical in shape and provided with a first cylindrical surface 202-a, a second cylindrical surface 202-b, and a bearing surface 202-c. As FIG. 17 also illustrates, the cylindrical surfaces 202-a, 202-b extend radially around an axis 202-d and are each provided with a diameter (referred to herein as a “hole diameter”). The hole diameter of the first cylindrical surface 202-a (referred to as the “first” hole diameter 202-e) is dimensioned according to the cylindrical shaft section 393 to provide a “close fit” between the first cylindrical surface 202-a and the cylindrical shaft section 393. The term “close fit” is used with respect to the cylindrical shaft section 393 and the hole 202 to refer to diameters of the cylindrical shaft section 393 and the hole 202 being dimensioned so that the axes of the cylindrical shaft section 393 and the hole 202 are maintained in alignment while, at the same time, allowing rotation and linear movement of the cylindrical shaft section 393 within the hole 202.

The extractor 100 is shown in FIGS. 59-62 provided with a mounting plate 200-a. The mounting plate 200-a is attached to the body 200 via a plurality of flat head cap screws 200-d, 200-e and tapped holes 200-f, 200-g. (In an alternative embodiment, however, the mounting plate 200-a is welded to the body 200.) The mounting plate 200-a provides the extractor 100 with a suitable structure upon which to mount at least a portion of the angle selector 70.

In the preferred embodiment, the mounting plate 200-a is shaped to cooperate with the shaft 390. As FIGS. 59-62 illustrate, the mounting plate 200-a is generally in the shape of a disc with a cylindrical surface 200-b defining the outside edge and a circular through-hole 200-c located at the center. The through-hole 200-c is provided with a diameter dimensioned to provide a close fit with the cylindrical shaft section 393 of the shaft 390. Thus, the through-hole 200-c is dimensioned so that the shaft 390 is firmly held therewithin with sufficient clearance that the shaft 390 moves axially and rotates within the through-hole 200-c.

The mounting plate 200-a is provided with a first side 200-d and a second side 200-e. The sides 200-d, 200-e extend radially from the through-hole 200-c to the cylindrical surface 200-b that defines the outer extent of the mounting plate 200-a. To facilitate attaching the mounting plate 200-a to the body 200, the mounting plate 200-a is provided with a cylindrical surface 200-f that extends across the second side 200-e and through the center of the mounting plate 200-a. The first side 200-d is shaped for the purpose of mounting the first gear 74 thereon. In the preferred embodiment, the mounting plate 200-a is shaped to orient the first gear 74 to be generally orthogonal to the axis 202-d of the hole 202 extending through the body 200. Thus, the first side 200-d of the mounting plate 200-a is substantially flat. The first side 200-d also acts as a stop that prevents the second gear 75 from moving axially beyond the plane of the first gear 74.

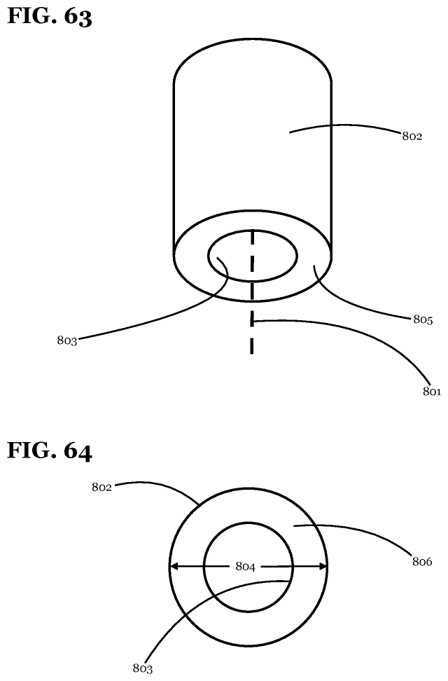

As FIGS. 2, 4, and 6 illustrate, the extractor 100 is provided with a tightener 800. As FIGS. 63 and 64 show, the tightener 800 includes an outer surface 802, an inner surface 803, and tightener bearing surfaces 805, 806 located at the extend ends of the tightener 800. As the foregoing implies, the outer and inner surfaces 802, 803 terminate at the tightener bearing surfaces 805, 806, which are annular in shape (but in an alternative embodiment are frusto-conically shaped).

The outer surface 802 is shaped to cooperate with the first leg opening 17 defined within the end 15 of the first leg section 11 of the bracket 10. The outer surface 802 is shaped so that the tightener 800 supports the bracket 10 (at least partially) while, at the same time, rotating within the first leg opening 17. In the preferred embodiment, the outer surface 802 of the tightener 800 is cylindrical about an axis 801 and provided with a diameter 804. The diameter 804 is dimensioned so that a close fit is achieved between the outer surface 802 of the tightener 800 and the first leg opening 17 of the bracket 10 so that the axes of the tightener 800 and the first leg opening 17 are maintained in alignment while at the same time allowing the tightener 800 to rotate and move axially within the first leg opening 17. The diameter 804 of the tightener 800 is also dimensioned so that a close fit is achieved between the outer surface 802 of the tightener 800 and the second cylindrical surface 202-b of the hole 202 extending through the body 200.

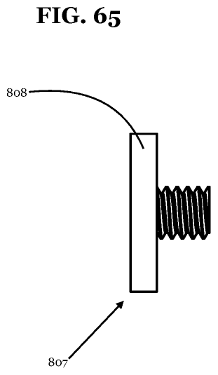

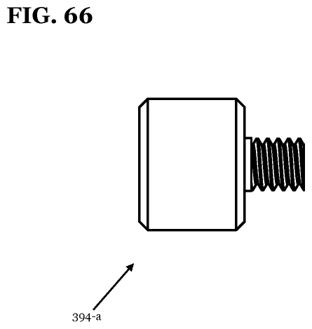

The inner surface 803 of the tightener 800 is provided with a thread profile that complements the threaded shaft section 395 and moves axially on the shaft 390 as the tightener 800 is rotated on the threaded shaft section 395. The tightener 800 is retained on the threaded shaft section 395 via the head 808 of a male threaded fastener 807 (shown in FIG. 65). As FIG. 11 illustrates, the male threaded fastener 807 is torqued into the first shaft hole 396 with the head 808 extending radially around the threaded shaft section 395 to act as a stop that retains the tightener 800 on the shaft 390.

As noted above, the first and second gears 74, 75 are in the form of a ring gear and a spur gear respectively. As a result, the teeth of the first gear 74 (the ring gear) extend radially inward toward the axis of rotation while the teeth of the second gear 75 (the spur gear) extend radially outward away from the axis of rotation. Thus, the teeth of the first and second gears 74, 75 mesh when the gears 74, 75 are co-planar but are disengaged when the gears 74, 75 are spaced axially (and therefore not co-planar).

The first gear 74 is provided with 180 internal teeth while the second gear 75 is provide with 180 external teeth. Thus, in the preferred embodiment, each tooth is radially positioned about the axis of rotation every 2 degrees, and hence, the teeth of the first and second gears 74, 75 mesh in 2-degree increments.

As noted above, the first gear 74 is fixed in place on the body 200 while the second gear 75 is attached to the shaft 390, which is rotatably secured within the hole 20 of the body 200. Thus, the teeth of the first gear 74 are rotatable with the shaft 390. As a result, when the teeth of the second gear are meshed with the teeth of the first gear 74, the first gear 74 prevents the second gear 75 from rotating (because the first gear 74 is fixed in place on the body 200). Conversely, when the teeth of the second gear 75 are disengaged from the teeth of the first gear 74, the second gear 75 is free to rotate with the shaft 390.

As noted above, the teeth of the first and second gears 74, 75 mesh when the first and second gears 74, 75 are co-planar but are disengaged when the first and second gears 74, 75 are spaced axially. Thus, by axially spacing the first and second gears 74, 75, the teeth are disengaged and the second gear 75 can rotate freely with the shaft 390. Conversely, by positioning the first and second gears 74, 75 to be co-planar, the teeth are meshed and the second gear 75 locked into place and rotational motion with the shaft 390 prevented.

As the foregoing illustrates, by moving the shaft 390 axially within the hole 202 of the body 200, the second gear 75 (which is attached to the shaft 390) is moved axially relative to the first gear 74, and therefore, the second gear 75 is positioned to be co-planar with the first gear 74 (and the teeth meshed) or spaced from each other axially (and the teeth no meshed). Thus, by moving the shaft 390 axially within the hole 202, the second gear 75 is positioned to rotate or remain fixed in place, thereby moving the angle selector 70 into the rotating configuration 77 or the locking configuration 78. For ease of reference, the distance between the locking configuration 78 (where the gears 74, 75 are co-planar) and the rotating configuration 77 (where the gears 74, 75 are rotating) shall be referred to as “axial gear spacing” and designated “79.”

As described above, the cylindrical shaft section 393 and the second gear 75 are axially aligned with the leg openings 17, 18 and secured between the leg sections 11, 12 of the bracket 10. As is also described above, the plate section 13 provides a spacing distance 19 between the leg sections 11, 12 of the bracket 10. The spacing distance 19 between the leg sections 11, 12 is dimensioned so that the bracket 10 moves the angle selector 70 axially between the rotating configuration 77 and the locking configuration 78, and hence, the spacing distance 19 between the leg sections 11, 12 is dimensioned to provide the extractor with axial gear spacing 79. In the preferred embodiment, first gear 74 and the second gear 75 are separated by axial gear spacing 79 that is at least the width of the first gear 74 (which measures ⅛ inches); however, in alternative embodiments, the first gear 74 is provided with a greater width, such as 10 mm.

By separating the second gear 75 axially from the first gear 74 by axial gear spacing 79, the bracket 10, and hence the plate section 13, is free to rotate about the first end 210 of the body 200. Thus, the plate section 13 is free to form an angle with respect to the axis 201 of the body 200. Then, by removing the axial gear spacing 79 separating the second gear 75 from the first gear 74, the gears 74, 75 are co-planar and the teeth of the second gear 75 are meshed with the teeth of the first gear 74. Because the first gear 74 is fixed in place to the body 200 and because the teeth of the second gear 75 (which is attached to the bracket) are meshed with the teeth of the first gear 74, the bracket 10 is fixed in place and cannot rotate about the first end 210 of the body. Because the bracket 10 cannot rotate about the first end 210 of the body, the plate section 13 of the bracket 10 cannot rotate either, and, as a result, the angle with respect to the axis 201 of the body 200 is fixed.

In the preferred embodiment, the second gear 75 is axially separated from the first gear 74 by simply pulling the second leg section 12 of the bracket 10 away from the body 200. Because the first gear 74 is fixed in place on the body 200 and because the second gear 75 is attached to the second leg section 12 of the bracket 10, the action of pulling the second leg section 12 away from the body 200 has the effect of axially separating the second gear 75 from the first gear 74 thereby placing the extractor 100 into the rotating configuration 77.

Conversely, the axial gear spacing between the gears 74, 75 is removed when the second leg section 12 of the bracket 10 is pushed toward the body 200. As noted above, because the first gear 74 is fixed to the body 200 while the second gear 75 attached to the second leg section 12 of the bracket 10, the action of pushing the second leg section 12 toward the body 200 has the effect of pushing the second gear 75 into being co-planar with the first gear 74 so that the gears 74, 75 mesh, thereby placing the extractor 100 into the locking configuration 78.

As FIGS. 50 and 53 illustrate, the second outer leg surface 12-b of the second leg section 12 of the bracket 10 is provided with an indicator 12-c. In the preferred embodiment, the indicator 12-c is pressed into the second outer leg surface 12-b; however, in an alternative embodiment, the indicator 12-c is engraved into the second outer leg surface 12-b. The indicator 12-c is oriented to be orthogonal to the plate section 13 of the bracket 10, and the second gear 75 is mounted to the second inner leg surface 12-a so a tooth is aligned with the indicator 12-c.

As FIG. 58 illustrates, the first gear 74 is provided with a plurality of marks 74-a, 74-b, which extend radially from the teeth of the first gear 74. The preferred embodiment is provided with two sets of marks 74-a, 74-b, a first set of marks 74-a wherein each of the marks 74-a is aligned with each tooth on the first gear 74 and a second set of a marks 75-b wherein each of the marks is aligned with every fifth tooth on the first gear 74. Because each of the gears 74, 75 has a tooth every two degrees around the circumference and because the first set of marks 74-a is aligned with each tooth, the first set of marks 74-a delineates two degrees of rotation while the second set of marks 74-b delineates ten degrees of rotation.

To maintain a desirable angle in a locked position, the tightener 800 on the threaded shaft section 395 is torqued so that the tightener 800 moves axially from the extreme end 398 of the shaft to a position where the tightener 800 bears against the bearing surface 202-c of the body 200. As the tightener 800 is being torqued, one of the tightener bearing surfaces 805, 806 bears against the bearing surface 202-c within the body 200. As the tightener 800 is torqued into the bearing surface 202-c of the body 200, the second gear 75 is clamped in place with its teeth meshed with the teeth of the first gear 74; thus, the shaft 390 is prevented from moving axially within the first cylindrical surface 202-a defined within the body 200. With axial motion thus prevented, axial gear spacing 79 cannot be created between the gears 74, 75. Consequently, the extractor 100 cannot be changed from the locking configuration 78 to the rotating configuration 77.

In sum, by pulling the second leg section 12 of the bracket 10 away from the body 200, the extractor 100 is placed into the rotating configuration 77 and the angle between the plate section 13 of the bracket 10 and the axis 201 of the body 200 can be changed. As the indicator 12-c on the second leg section 12 of the bracket rotates past each of the marks 74-a, 74-b on the first gear 74, the angle between the plate section 13 of the bracket 10 and the axis 201 of the body 200 and the plate section 13 of the bracket 10 is changed by two degrees. By aligning the indicator 12-c with one of the marks 74-a, 74-b on the second gear 75 and by pushing the second leg section 12 of the bracket 10 toward the body 200, a desirable angle between the plate section 13 and the axis of the body 200 can be selected and locked into place.

As described above, when the upper wedging surfaces 216, 218 of the body 200 are positioned under the trunnion 1102 and extend in a generally parallel orientation relative to the bottom surface 1104, the axis 201 of the body 200 is generally parallel to the axis 1101 of the trunnion 1102. As a result, when the plate section 13 of the bracket 10 is oriented at an angle relative to the axis 201 of the body 200, the plate section 13 is also oriented at the same general angle relative to the axis 1101 of the trunnion 1102.

Because surgeons practicing joint replacement are quite familiar with the angle 1411 between the trunnion axis 1101 and the impacting axis 1401 and the angle 1211 between the trunnion axis 1101 and the stem axis 1201 of a given femoral component, the indicator 12-c also shows the angle between the plate section 13 and the impacting axis 1401 of the femoral component 1000 and the angle between the plate section 13 and the stem axis 1201 of the femoral component 1000.

Consequently, the plate section 13 (and the strike plate 500 attached thereto) can be oriented so that, when the strike plate 500 is impacted, the direction of the impulse transmitted to the femoral component 1000 can be controlled. Thus, the plate section 13 and the strike plate 500 can be oriented so that an impact is transmitted to the femoral component 1000 in a direction that is advantageous to extracting the femoral component 1000 from the patient's femur.

By way of example and not limitation, it may be desirable to orient the plate section 13 and the strike plate 500 to be orthogonal to the impacting axis 1401 so that when the strike plate 500 is impacted, the impulse delivered to the femoral component 1000 is in a direction pointing out of the femur. Those with skill in the art will appreciate that the plate section 13 can be rotated into a plurality of orientations so that the femoral component 1000 is impacted at different angles that are advantageous to breaking the bonds of osseointegration.

While this invention has been particularly shown and described with references to preferred embodiments thereof, it will be understood by those skilled in the art that various changes in form and details may be made therein without departing from the spirit and scope of the invention as defined by the appended claims.

As FIG. __ illustrates, the first gear 75 is provided with

It is preferred that the femoral component 1000

As noted above, the spacing distance 19 is dimensioned so that the bracket 10 can move the second gear 75 from being axially spaced from the first gear 75 to be co-planar with the first gear 74. The spacing distance 19 is also dimensioned so that the Thus, the plate section 13 of the bracket 10 forms a predetermined angle with the axis 201 of the body 200 when the bracket-shaft subassembly is rotated within the hole 202 of the body 200.
a body 200 with a pivoting member 300 and a strike plate (preferably a plurality of strike plates 501, 502, as is shown in FIG. 1). A pivot 400 in the form of a stainless steel pin secures the pivoting member 300 to the body 200. The body 200, the pivoting member 300, the strike plates 501, 502, and the pivot 400 are preferably manufactured from a stainless steel, such as the 300 or 400 grade stainless steels (e.g. 304, 316, and 416 stainless steel); however, in an alternative embodiment, the body 200, the pivoting member 300, the strike plates 501, 502, and the pivot 400 are manufactured from titanium.

Each of the strike plates 501, 502 (referred to as a “first strike plate 501” and a “second strike plate 502” to distinguish one from the other) is provided with an upper striking surface (designated 501a and 502a) and a lower striking surface (designated 501b and 502b). Also shown in FIG. 1, the extractor 100 is provided with a first end 600 and a second end 700, as well as a plurality of extractor sections 110, 120, 130, each of which includes an axis designated 111, 121, 131 respectively. FIG. 1 depicts the extractor 100 with a first section 110 (also referred to as a “clamping section 110”) with a first axis 111 (also referred to as a “clamping axis 111”), an upper extractor section 120 with an upper axis 121, and a central extractor section 130 with a central axis 131.

As FIG. 1 illustrates, the upper axis 121 and the clamping axis 111 are generally parallel in orientation. As FIG. 1 also shows, the first end 600 terminates at an opening 601 that is formed by the pivoting member 300 and the body 200, while the second end 700 terminates at the second strike place 502. FIG. 1 further depicts the extractor 100 provided with a threaded component 112 that engages with internal threads located within the body 200. In the preferred embodiment, the threaded component 112 is a hex bolt (and therefore is provided with threaded shank and a torque transferring structure in the form of a hex head). One of ordinary skill in the art will appreciate that a hex bolt is not necessary; in an alternative embodiment, the extractor 100 is provided with a threaded T-bar wherein the torque transferring structure is a T-handle.

Referring now to FIG. 2, the body 200 of the extractor 100 is shown. As depicted therein, the body 200 is provided with first and second ends 800, 900 respectively. The first and second ends 800, 900 of the body 200 generally correspond to the first and second ends of the extractor 100. The body 200 is provided with an angled section, preferably a plurality of angled section; accordingly, the body shown in FIG. 2 includes a first angled section, more specifically referred to herein as a clamping body section 210, and a second angled section 220, more specifically referred to herein as an impacting body section. Located between the angled sections 210, 220, a central body section 230 is included within the body 200. As FIG. 2 also shows, each of the section is provided with an axis. The clamping body section 210 is provided with a clamping body axis 211; the impacting body section 220 is provided with an impacting body axis 221, and the central body section 230 is provided with a central body axis 231. As is evident in the figures, the clamping body axis 211 and the impacting body axis 221 are generally parallel to each other.

In the preferred embodiment, the body 200 is generally cylindrical in shape (and hence provided with a generally circular cross-sectional shape). However, one of ordinary skill in the art will appreciate that other cross-sectional shapes are within the scope of the present invention. By way of example (and not limitation), it is permissible for the body to be rectangular in shape (and hence have a square or rectangular cross-sectional shape). In an alternative embodiment, the body 200 is fabricated from hexagonal bar stock, which provides a hexagonal cross-sectional shape. In such an embodiment, the body 200 is provided with a polygonal cross-sectional shape. Thought he preferred embodiment is fabricated from round bar stock, alternative embodiments are fabricated from semi-circular bar stock.

Much like the extractor 100, the various sections 210, 220, 230 of the body 200 are each provided with an axis. FIG. 2 illustrates that the body 200 is provided with a plurality of axes, including a central body axis 231, an impacting body axis 221, and a clamping body axis 211.

FIG. 3 depicts the first section 210 of the body 200 in greater detail. As shown therein, the first section is provided with a plurality of through holes, including a pivot hole 401 and a threaded hole 413. The pivot hole 401 and the threaded hole 413 are oriented to be generally orthogonal to each other. The first end 210 of the body 200 is milled to provide a fulcrum structure 410 with an angled fulcrum surface 411. The angled fulcrum surface 411 extends from the pivot hole 401 in a direction that is both radially inward towards the axis 11 of the extractor 100 (shown in FIG. 1) and axially towards the second end 220 of the body 200 so that the fulcrum surface 411 is oriented at an angle 414 (shown in FIG. 7) relative to the axis 111 of the first extractor section 110. In the preferred embodiment, this angle 414 measures 20 degrees relative to the axis 111.

The angled fulcrum surface 411 terminates (at least in part) at a tightening surface 412. The tightening surface 412 extends radially inward towards the axis 111 of the first end 110 of the extractor 100 and axially away from the second end 220 of the body 200 so that the plane of the tightening surface 412 is at an angle relative to the clamping body axis 211 that measures 20 degrees. The tightening surface 412 terminates where the body 200 provides a trunnion accepting structure 2102.

The trunnion accepting structure 2102 is provided with an arm extension bar 214 and a pair of arms 212, 213. From where the tightening surface 412 terminates, the arm extension bar 214 extends axially so as to accommodate the axial dimension of the trunnion of the femoral component between the tightening surface 412 and the arms 212, 213. Each of the arms 212, 213 extends from the arm extension bar 214 so that each is generally parallel to the other. The arms 212, 213 are spaced from each other so as to define a notch 215 dimensioned according to a diameter of the trunnion of the femoral component; in the embodiment shown herein, the arms 212, 213 are spaced less than 0.55 inches, preferably between 0.5 inches and 0.375 inches, from each other, inclusively.

As FIG. 3 also illustrates, the arms 212, 213 are provided with upper arm surfaces 216, 218 and lower arm surfaces 217, 219. The upper arm surfaces 216, 218 are spaced axially from the pivot hole 401 at distance designated “D” in FIG. 3. In the preferred embodiment, “D” measures greater than 0.551 inches.

FIG. 4 depicts a view of the lower arm surfaces 217, 219 extending from the arm extension bar 214 and defining the notch 215 thereinbetween. The notch 215 is dimensioned to accept, at least in part, the femoral component, preferably the neck of the femoral component.

Referring now to FIGS. 5 and 6, perspective views of the pivoting member 300 are shown. As both FIGS. 5 and 6 show, the presently preferred pivoting member 300 is in the shape of a cylinder, preferably a half cylinder, with the cylindrically-shaped profile enclosing a pocket 302 and an axis 301 (referred to as the “pivoting axis 301”). The pocket 302 terminates to form a clamping structure 303. As FIG. 6 illustrates, it is preferred that the clamping structure 303 be in the form of a tooth that engages the neck of the femoral component.

In the preferred embodiment, the pivoting member is fabricated from semi-circular bar stock with the pocket 302 milled out through the use of a ball-nose end mill. The hole 401 for the pivot 400 is simply drilled using an appropriately sized drill for a stainless steel pin. While the preferred embodiment is cylindrically-shaped, those with ordinary skill in the art will appreciate that other shapes are within the scope of the present invention. For example, in an alternative embodiment, the pivoting member 300 is fabricated by milling the pocket 302 into rectangular bar stock thereby yielding a pivoting member 300 that is rectangular in shape. In yet another alternative embodiment, the pivoting member 300 is fabricated by milling the pocket 302 from hexagonal bar stock, thereby yielding a pivoting member 300 that is polygonal in shape.

As noted above (and as FIG. 6 illustrates), the clamping structure 303 is in the form of a tooth that is between 0.0625″ and 0.25″ inches wide inclusively (with the preferred width being 0.125″). Though the clamping structure 303 is in the form of a tooth, those with skill in the art will appreciate that the clamping structure 303 can take other forms and dimensions.

Turning to FIG. 7, the pivoting member 300 is attached to the body 200 via a pivot 400, which, as noted above, is in the form of a stainless steel pin. FIG. 7 depicts a cross-sectional view of the extractor 100 with the pivoting member 300 so attached. As noted above and as FIG. 7 shows, the fulcrum structure 410 of the body 200 includes the angled fulcrum surface 411 and the tightening surface 412. Consequently, when the pivoting member 300 is attached to the body 200, the pivoting member 300 (and hence the axis 301 of the pivoting member 300) is rotatable on the fulcrum structure 410 of the body 200. Thus, the pivoting member 300 is rotatable so that the axis 301 of the pivoting member 300 is generally parallel with the fulcrum surface 411 (such as when the threaded component 112 is retracted) or generally parallel with the tightening surface 412 (such as when the threaded component 112 is tightened and torqued into the body 200. As noted above, the range of rotation of the pivoting member 300 between the fulcrum surface 411 and the tightening surface 412 is 40 degrees.

As noted above, the extractor 100 is configured to remove a femoral component from a patient's femur during a hip revision.

As noted above, the body 200 of the extractor 100 is configured to remove from a patient's femur the femoral component (such as the standard femoral component 1000 depicted in FIGS. 8, 9, and 10). FIGS. 11 and 12 illustrate how the body 200 of the extractor 100 is configured to remove the femoral component. FIG. 11 shows the notch 215 of the body 200 accepting at least a portion of the trunnion neck 1300. The arms 212, 213 of the body 200 are dimensioned so that they extend around at least a portion of the trunnion neck 1300 and the upper arm surfaces 216, 218 in contact with the bottom surface 1104 of the trunnion 1102. As FIGS. 11 and 12 illustrate, the arm extension bar 214 extends axially so as to accommodate the axial dimension of the trunnion 1104 between the tightening surface 412 and the upper arm surfaces 216, 218. Thus, the trunnion accepting structure 2102 is configured to accommodate at least a portion of the trunnion 1104.

Turning now to FIGS. 13, 14, and 15, the extractor 100 is shown configured to accept the trunnion 1102 of the femoral component 1000. FIG. 13 is a perspective view of the extractor 100 after the pivoting member 300 has been rotated about the pivot 400 so that the axis 301 is generally parallel with the plane of the fulcrum surface 411. FIG. 14 depicts a sectional view of the extractor 100 shown in FIG. 13. FIG. 15 is a perspective view of the opening 601 that is formed when the pivoting member 300 is rotated about the pivot 400 so that the axis 301 is generally parallel with the plane of the fulcrum surface 411. As noted above, and as FIGS. 13, 14, and 15 illustrate, the fulcrum structure 410 is shaped to enable the pivoting member 300 to be rotated so that the axis 301 of the pivoting member 300 approaches a position that is generally parallel with the plane of the fulcrum surface 411. By thus rotating the pivoting member 300 towards the plane of the fulcrum surface 411, the opening 601 is increasingly enlarged to accommodate trunnions of increasing size. Thus, the extractor 100 is provided with an opening 601 that is configured to accept the trunnion 1102 of virtually any femoral component 1000.

After the pivoting member 300 is rotated about the pivot 400 so that the axis 301 is generally parallel with the plane of the fulcrum surface 411, the extractor 100 is “opened” so that the trunnion 1102 easily fits through the opening 601. FIGS. 16 and 17 depict the extractor 100 thus opened and accommodating a trunnion 1102 through the opening 601. As FIGS. 16 and 17 demonstrate, the arms 212, 213 are positioned relative to the trunnion 1102 so that the upper arm surfaces 216, 218 contact at least a portion of the bottom surface 1104 of the trunnion 1102. As FIGS. 16 and 17 further show, the trunnion neck 1300 is located within the notch 215 between the arms 212, 213. With the trunnion 1102 thus disposed within the extractor, a force can be exerted upon the bottom surface 1104 of the trunnion 1102.

Referring now to FIGS. 18 and 19, the extractor 100 is shown configured to clamp the trunnion neck 1300 securely. After the arms 212, 213 are positioned under the trunnion 1102, it is desirable to torque the threaded component 112 into the threaded hole 413 of the body 200 so as to clamp the femoral component 1000 about the trunnion neck 1300. The threaded component 112 is provided with a torque transferring structure 113 in the form of a hex head and a threaded shank 114. The threaded component 112 is dimensioned so that at least a portion of the threaded shank 114 extends through the threaded hole 413 and contacts the pivoting member 300, preferably the pocket 302. Once the threaded component 112 contacts the pivoting member 300, continued torqueing of the threaded component 112 into the body 200 will exert a force, designated “Fx” in FIG. 18, on the pivoting member 300 at the point of contact. The force, Fx, exerted by the threaded component 112 as it is torqued into the body 200 will cause the pivoting member 300 to rotate about the pivot 400 so that the axis 301 of the pivoting member 300 approaches the plane of the tightening surface 412. As the axis 301 of the pivoting member 300 approaches the plane of the tightening surface 412, the pivoting member 300 contacts the trunnion neck 1300 and, at the point of contact, exerts a force of equal magnitude, but in the opposite direction, as the force applied by the threaded component 112; the force that the pivoting member 300 exerts on the trunnion neck 1300 is designated Fx′ (“Fx prime”) on FIG. 18. At the point of contact between the pivoting member 300 and the trunnion neck 1300, the pivoting member is provided with the clamping structure 303, as FIGS. 18 and 19 illustrate. Thus, the threaded component 112 uses the threads of the body's threaded hole 413 as leverage to force the clamping structure 303 onto the trunnion neck 1300. As the axis 301 of the pivoting member 300 approaches the plane of the tightening surface 412, the force, Fx′, that the clamping structure 303 exerts on the trunnion neck 1300 increases.

In the preferred embodiment, the clamping structure 303 of the pivoting member 300 is dimensioned so that it fits, at least in part, under the trunnion 1102. As noted above, the pivoting member 200 rotates about the pivot 400, and therefore, the inner tooth surface 304 moves in a circle about the pivot 400 with a radius, designated “R” on FIG. 18, that is greater than 0.779 inches. The radius R is dimensioned according to the axial distance between the pivot hole 401 and the upper arm surfaces 216, 218 (which is designated “D” in FIG. 3). The relationship between the dimensions of radius “R” (shown in FIG. 18) and axial distance “D” (shown in FIG. 3) is the following: R²=(0.799)²+D². Thus, the radius R is dimensioned according to the axial distance “D” separating the upper arm surfaces 216, 218 and the pivot hole 401.

As noted above, the inner tooth surface 304 rotates about the pivot 400 at a radius R, and therefore, the clamping structure 303 of the pivoting member 300 also rotates about the pivot 400 at a radius R. As noted above, the radius R is dimensioned so that the clamping structure 303 contacts the trunnion neck 1300 at a location that is generally co-planar with at least one of the arms 212, 213, as FIGS. 18 and 19 depict. The term “generally co-planar” as used in this context means within 1 millimeter of the plane of one of the arms. Thus, the pivoting member 300 is dimensioned so that the clamping structure 303 cooperates with the arms 212, 213 of the body 200. As the threaded component 112 is torqued into the threaded hole 413, the inner tooth surface 304 is forced under the trunnion 1102 and into contact with the bottom surface 1104 of the trunnion 1102; the trunnion neck 1300 is also forced between the arms 212, 213 and into the notch 215, thereby bringing the bottom trunnion surface 1104 into increasing contact with the upper arm surfaces 216, 218 of the arms 212, 213.

Various figures provided herein disclose that the body 200 is provided with a plurality of strike plates 501, 502. The strike plates 501, 502 are oriented to extend from the body 200 so that at least one of the strike plates 501, 502 is generally orthogonal to the impacting axis 1401 of the femoral component 1000. Inn the preferred embodiment, both of the strike plates 501, 502 and both of the lower striking surfaces 501b, 502b are oriented to be orthogonal to the impacting axis 1401 of the femoral component 1000. Thus, a surface of at least one of the strike plates 501, 502 is oriented to be generally orthogonal relative to an axis of the femoral component. In an alternative embodiment, at least one of the striking surfaces 501b, 502b is oriented to be orthogonal to the impacting axis 1401 of the femoral component 1000. In such an alternative embodiment, the lower striking surface 501b of the first strike plate 501 is oriented to be generally orthogonal relative to the impacting axis 1401 while the lower striking surface 502b of the second strike plate 502 is oriented to be orthogonal to the stem axis 1201, or the lower striking surface 502b of the second strike plate 502 can be oriented to be orthogonal relative to the impacting axis 1401 while the lower striking surface 501b of the first strike plate 501 can be oriented to be orthogonal relative to the stem axis 1201. In yet another alternative embodiment, both of th lower striking surfaces 501b, 502b are oriented to be generally orthogonal to the stem axis 1201 (rather than the impacting axis 1401 as is presently preferred). Thus, the strike plates 501, 502, and the surfaces on the strike plates 501, 502 are oriented so that an impact is imparted to the femoral component in the direction of an axis of the femoral component.

As noted above, the clamping section 110 of the extractor 100 is provided with an axis 111. As FIG. 19 illustrates, the axis 111 of the extractor's clamping section 110 is generally parallel to the trunnion axis 1101. Because the bottom surface 1104 of the trunnion 1102 is generally orthogonal to the trunnion axis 1101, and because the axis 111 of the clamping section 110 is generally parallel with the trunnion axis 1101, the bottom surface 1104 of the trunnion 1102 is generally orthogonal to the axis 111 of the clamping section 110 of the extractor 100. Thus, the clamping section 110 of the extractor is oriented according to the trunnion axis 1101 of the femoral component.

As is also noted above, the extractor 100 is provided with a central extractor section 130 and a central axis 131. Similarly, the body 200 is also provided with a central body section 230 with a central body axis 231. Both the central axis 131 of the extractor 100 and the central body axis 231 are generally co-axial, as are the axes 111, 211 of the extractor 100 and the extractor body 200. The central axes 131, 231 are oriented at an angle relative to the axes 111, 211 of the extractor's clamping section 110 and the body's clamping section 210 that is substantially equal to the angle between the trunnion axis 1101 that is equal in to the angle between the trunnion axis 1101 and the impacting axis 1401. In the embodiment shown herein, this angle ranges between 130 and 150 degrees inclusively, preferably 130 degrees. Thus, the angle between the central axis 131 and the axis 111 of the clamping section 110 is substantially the same as the angle between the trunnion axis 1101 and the impacting axis 1401. One of ordinary skill in the art will appreciate that the angle between the axes 111, 211 of the central sections of the extractor 100 and the body 200 respectively are can be substantially equal to the angle between the trunnion axis 1401 and the stem axis 1201 without departing from the spirit of the present invention. As used in the context of the angles between the various axes of the extractor and the body, the term “substantially” means within a margin of variance in manufacturing and use that is 10%.

One of ordinary skill in the art will understand that the extractor 100 can be used with a slap hammer or a mallet. A slap hammer (not shown) can be attached to one of the strike places 501, 502 such as by screwing a slap hammer shaft into an internal thread tapped into the plates 501 502. In such an arrangement, the slap hammer extends from one of the upper striking surface 501a, 502a at an angle that is generally orthogonal to the strike plates 501, 502 (and hence parallel to the orientation of the impacting axis 1401 of the femoral component 1000). Thus, when the slap hammer is employed, the femoral component 1000 is removed from a patient's femur at substantially the same angle as it was inserted. A mallet can be used by striking one of the lower striking surfaces 501b, 502b and removing the femoral component 1000 at substantially the same angle as it was originally impacted into the femur.

Though the preferred embodiment is provided with upper striking surfaces 501a, 502a and lower striking surfaces 501b, 502b that are generally parallel with each other and generally orthogonal to the impacting axis 1401 of the femoral component 1000, alternative embodiments are provided with upper striking surfaces 501a, 502a and lower striking surfaces 501b, 502b that are generally orthogonal to the stem axis 1201. In yet another alternative embodiment, one of the strike plates 501, 502 is orthogonal to the impacting axis 1401 while the other is orthogonal to the stem axis 1201. In such an arrangement, a mallet can be used to strike the strike plate that is generally orthogonal to the stem axis 1201 while a slap hammer is attached to the other strike plate at an angle that is orthogonal to the impacting axis 1401. One of ordinary skill in the art will appreciate that the foregoing arrangement can be reversed with the slap hammer attached to one of the strike plates 501, 502 at an angle that is generally orthogonal to the stem axis 1201 while the other strike plate is oriented at an angle that is generally orthogonal to the impacting axis 1401.

FIGS. 20 and 21 depict an alternative embodiment of the present invention.

As shown therein, the extractor 100 includes a first section 110 (also referred to as a “clamping section 110”), a second section 120 (also referred to as an “upper section”), and a third section 130 (also referred to as a “central section”). The extractor is also provided with a body 200, a pivoting member 300, and a pivot 400.

The body 200 of the alternative embodiment shown in FIG. 20 is depicted in FIG. 22 and is largely the same as the body 200 disclosed as the presently preferred embodiment. As shown in FIG. 22, the body 200 is provided with a first body section 210 (also referred to as the “clamping body section 210”), a second body section 220 (also referred to as the “impacting body section 220”) and a third body section 230 (also referred to as the “central body section 230”). However, unlike the pivoting member 300 in the preferred embodiment, the pivoting member 300 of the alternative embodiment shown in FIGS. 20 and 21 extends substantially the entire length of the body 200 and terminates within the upper section 120 of the extractor 100 under the second strike plate 502. Additionally, unlike the preferred embodiment, the pivot hole 401 in FIGS. 20 and 21 is defined within the central body section 230, and the threaded hole 413 is defined within the upper body section 220.

As FIG. 20 depicts, the pivoting member 300 is provided with a pivoting angle 310. By the virtue of the pivoting angle 310, a surgeon can squeeze the upper section 120 of the extractor 100 and thereby force the pivoting member 300 and the body 200 together at the upper section 120. By forcing the pivoting member 300 and the body together at the upper section 120, the surgeon forces the clamping structure 303 away from the arms 212 of the body 200 at the opening 601. Thus, the opening 601 of the extractor 100 is enlarged.

Much like the preferred embodiment, the alternative embodiment shown in FIGS. 20, 21, and 22 is provided with a threaded component 112 that is dimensioned so that at least a portion of the threaded shank 114 extends through the threaded hole 413 and contacts the pivoting member 300, preferably the pocket 302. Once the threaded component 112 contacts the pivoting member 300, continued torqueing of the threaded component 112 into the body 200 in the direction of Fx will cause the pivoting member 300 to rotate about the pivot 400 (counter clockwise as shown in FIG. 20) so that the clamping structure 304 rotates towards the trunnion neck 1300. Thus, as the threaded component 112 is torqued into the body 200, the pivoting member 300 exerts a greater clamping force on the trunnion neck 1300. Much like the preferred embodiment, in the alternative embodiment shown in FIGS. 20, 21, and 22 the threaded component 112 uses the threads of the body's threaded hole 413 as leverage to force the clamping structure 303 onto the trunnion neck 1300.

While this invention has been particularly shown and described with references to preferred embodiments thereof, it will be understood by those skilled in the art that various changes in form and details may be made therein without departing from the spirit and scope of the invention as defined by the appended claims.

What is claimed is:

1: An extractor for a femoral component with a trunnion neck comprising,
a) a plurality of extractor sections, including a first section with a first axis, a second section with a second axis, and a third section with a third axis;
b) a body with a threaded hole defined therein that is provided with a clamping body section and a central body section wherein:
i) the clamping body section is provided with a fulcrum structure with a pivot hole and a threaded hole defined therein, a fulcrum surface, and a tightening surface;
ii) the fulcrum surface extends from the pivot hole in a direction that is both radially inward towards the first axis of the first extractor section and axially towards the central body section so that the fulcrum surface is oriented at an angle relative to the first axis of the first extractor section;
iii) the threaded hole is generally orthogonal relative to the pivot hole of the body;
c) a pivoting member with first end, a second end, and a pivot hole defined thereinbetween that includes a clamping structure located at the second end that is shaped to clamp the trunnion neck of the femoral component;
d) a pivot that secures the pivoting member to the body by extending through the pivot hole defined in the pivoting member and the pivot hole defined in the fulcrum structure of the body;
e) a threaded member with a shank that is has been torqued into the threaded hole of the body; and
f) the shank of the threaded member is dimensioned to extend through the threaded hole of the body and contact the pivoting member at the first end.

2: An extractor according to claim 1 wherein the extractor is fabricated from a stainless steel.

3: An extractor for a femoral component with a trunnion neck comprising,
a) a plurality of extractor sections, including a first section with a first axis, a second section with a second axis, and a third section with a third axis;
b) a body that is provided with a first body section, a central body section, and an upper body section wherein:
i) the central body section defines a pivot hole therein;
ii) the upper body section defines a threaded hole that is generally orthogonal relative to the pivot hole of the central body section;
c) a pivoting member with first end, a second end, a pivot hole defined between the first end and the second end, that includes a pivoting angle and a clamping structure located at the second end that is shaped to clamp the trunnion neck of the femoral component;
d) a pivot that secures the pivoting member to the body by extending through the pivot hole defined in the pivoting member and the pivot hole defined in the central body section;
e) a threaded member with a shank that is has been torqued into the threaded hole of the body; and
f) the shank of the threaded member is dimensioned to extend through the threaded hole of the body and contact the pivoting member at the first end.

4: An extractor according to claim 3 wherein the extractor has been fabricated from a stainless steel.
